SEQUENCE LISTING (1 sequence)

SEQ ID NO: 1 - protein, 14 aa, Artificial Sequence
  Peptide LP3
GDNDSEDNSDEENC

(19) United States
(12) Patent Application Publication
(10) Pub. No.: US 2022/0220155 A1
(43) Pub. Date: Jul. 14, 2022

(54) VASCULAR CHOLESTEROL INHIBITORS AND USE THEREOF

(71) Applicant: CONSEJO SUPERIOR DE INVESTIGACIONES CIENTIFICAS, Madrid (ES)

(72) Inventors: María Teresa TARRAGÓ CLUA, Barcelona (ES); Roger PRADES COSANO, Barcelona (ES); Chiara PALLARA, Barcelona (ES); Vicenta LLORENTE CORTÉS, Barcelona (ES)

(21) Appl. No.: 17/609,118

(22) PCT Filed: May 5, 2020
(86) PCT No.: PCT/EP2020/062416

§ 371 Date: Nov. 5, 2021

(30) Foreign Application Priority Data
May 6, 2019 (EP) 19382335.8

(51) Int. Cl. C07K 7/08; C07K 7/06; A61P 3/06

(52) U.S. Cl. CPC C07K 7/08; A61K 38/00; A61P 3/06; C07K 7/06

(57) ABSTRACT

The present invention relates to compounds having pharmacological activity in the treatment of vascular cholesterol accumulation, plasma low-density lipoproteins (LDL) abnormal aggregation and/or inhibition or reduction of the formation of VSMC foam cells, processes for their preparation, pharmaceutical compositions comprising them, and their use in therapy and/or prophylaxis of conditions wherein decrease of vascular cholesterol accumulation, inhibition of LDL aggregation, inhibition or reduction of the formation of VSMC foam cells and/or prevention of aggregated LDL (agLDL) internalization is useful such as atherosclerosis, coronary artery disease, stroke, peripheral artery disease, angina pectoris, thrombosis, hyperlipidemia, hyperlipoproteinemia type II, familial hypercholesterolemia, familial combined hyperlipidemia, type II diabetes, hypothyroidism, Cushing's syndrome and obesity.

### FIELD OF THE INVENTION

The present invention relates to compounds having pharmacological activity in the treatment of vascular cholesterol accumulation and plasma low-density lipoproteins (LDL) abnormal aggregation, to processes of preparation of such compounds, to pharmaceutical compositions comprising them and their use in therapy and/or prophylaxis of conditions wherein decrease of vascular cholesterol accumulation, inhibition of LDL aggregation and/or prevention of aggregated LDL (agLDL) internalization is useful, such as atherosclerosis and all the atherosclerotic cardiovascular diseases (ASCVD) (e.g., coronary artery disease, stroke, peripheral artery disease, angina pectoris, thrombosis) as well as hypercolesterolemic conditions and/or abnormalities in lipoprotein metabolism (e.g. hyperlipidemia, hyperlipoproteinemia type II, familial hypercholesterolemia, familial combined hyperlipidemia, type II diabetes, hypothyroidism, Cushing's syndrome, obesity).

### BACKGROUND

Cardiovascular (CV) disease is the leading cause of mortality worldwide, causing about 31.4% of deaths in 2012. (World Health Organization: Health statistics and information systems. Cause-specific mortality. Glob Summ estimates for 2000-2012. Available at: “http://www.who.int/healthinfo/globalburdendisease/estimates/en/indexl.html” Accessed Jan. 2, 2015.) Data from 2010 demonstrate that CV disease accounted for 31.9% of US deaths, with ischemic heart disease and stroke leading to the vast majority (total 27.6%; 21.1%, and 6.5%, respectively) and generated a resulting global cost estimate of $863 billion in 2010, with a 22% increase expected by 2030. (Bloom, D. E., Cafiero, E. T., Jane-Llopis, E., Abrahams-Gessel, S., Bloom, L. R., Fathima, S., Feigl, A. B., Gaziano, T., Mowafi, M., Pandya, A., Prettner, K., Rosenberg, L., Seligman, B., Stein, A. Z., & Weinstein, C. (2011) The Global Economic Burden of Noncommunicable Diseases. Geneva: World Economic Forum (http://www3.weforum.org/docs/WEF_Harvard_HE_GlobalEconomicBurdenNonCom municableDiseases_2011.pdf).

A large, worldwide study demonstrated that among all modifiable risk factors, abnormal lipid levels were associated with the highest population attributable risk (approximately 50%) for the occurrence of myocardial infarction (MI) (Yusuf S et al. Lancet. 2004 Sep. 11-17; 364(9438):937-52 [PMID 15364185]). Similarly, the Framingham heart study showed that cardiovascular risk positively correlates with blood levels of LDL-cholesterol and inversely with HDL-cholesterol (Castelli W P et al. Ann Epidemiol. 1992 January-March; 2(1-2):23-8 [PMID 1342260] and Gordon T et al. Am J Med. 1977 May; 62(5):707-14 [PMID 193398]. Accordingly, in western countries, lifestyle interventions and evidence-based therapies, including those focused on hypercholesterolemia, have led to a reduction in CV risk at a population level. In a series of studies covering the 1980 to 2010 time period in the United States, Canada and Europe, it was estimated that 19%-46% of the total reduction in the rate of coronary heart disease (CHD) mortality was explained by a reduction in total cholesterol levels attributed to lifestyle changes and pharmacological treatment. (Bjorck L. et al. Eur Heart J. 2009 May; 30(9):1046-56 [PMID 19141562], Bandosz P. et al. BMJ. 2012 January 25; 344:d8136. [PMID 22279114], Wijeysundera H C. et al. JAMA. 2010 May 12; 303(18):1841-7 [PMID 20460623], Flores-Mateo G. Rev Esp Cardiol. 2011 November; 64(11):988-96 [PMDI 21962958], Hughes J. et al. Eur J Prev Cardiol. 2013 April; 20(2):310-21 [PMID 22403395], Ford E S. et al. N Engl J Med. 2007 Jun. 7; 356(23):2388-98 [PMID 17554120], Aspelund T. et al. PLoS One. 2010 Nov. 12; 5(11):e13957 [PMID 21103050], Palmieri L. et al. Am J Public Health. 2010 April; 100(4):684-92 [PMID 19608958], Bajekal M et al. PLoS Med. 2012; 9(6):e1001237 [PMID 22719232] and Hotchkiss J W et al. BMJ. 2014 Feb. 6; 348:g1088 [PMID 24503058]).

Circulating blood cholesterol is mainly carried by low density lipoprotein (LDL) particles, which are thus key players in cholesterol transfer and metabolism from liver to any body cell (Hevonoja T. et al. Biochim Biophys Acta. 2000 Nov. 15; 1488(3):189-210 [PMID 11082530]). Circulating LDL particles are able to penetrate the endothelium of the arterial walls and became oxidized and aggregated, promote inflammation, and drive injury to the overlying endothelium and surrounding smooth muscle cells (Ross R. et al. N Engl J Med. 1999 Jan. 14; 340(2):115-26 [PMID 9887164]).

Persistent elevation in circulating LDL-cholesterol has been directly linked to alterations in the endothelium permeability that in turn promotes LDL influx into the arterial intima (Guretzki H J. et al. Atherosclerosis. 1994 May; 107(1):15-24 [PMID 7945555]). For example, LDL receptor-deficient mice (unable to clear LDL from the circulation) have elevated LDL-cholesterol and consequently develop severe atherosclerosis (Véniant MM. et al. J Clin Invest. 2000 December; 106(12):1501-10 [PMID 11120757]). Conversely mice with virtually no LDL-cholesterol do not develop atherosclerosis regardless of diet and other heart disease risk factors (Lieu H D. et al. Circulation. 2003 Mar. 11; 107(9):1315-21 [PMID 12628954]). Epidemiological investigations have validated LDL-cholesterol blood level as an independent predictor of CV risk. According to the Framingham Heart Study, people exhibiting LDL-cholesterol level higher than 160 mg/dL were more likely to develop clinically significant CHD compared to a reference population with LDL-cholesterol lower than 130 mg/dL. (Wilson P W. et al. Circulation. 1998 May 12; 97(18):1837-47 [PMID 9603539]). Similarly, in the Atherosclerosis Risk in Communities (ARIC) study was demonstrated that the risk of an incident CHD event was elevated by approximately 40% for every 39 mg/dL incremental increase in LDL-cholesterol (Sharrett A R. et al. Circulation. 2001 Sep. 4; 104(10):1108-13 [PMID 11535564]).

Together with abnormal LDL-cholesterol levels, alternative criteria related to the atherogenic lipoproteins have been demonstrated to be equally relevant CV risk factors. For example, it has been demonstrated that at identical LDL-cholesterol levels, measurement of the number of circulating LDL particles has important prognostic value since higher number of smaller LDL particles is associated with higher CV risk (Otvos J D. et al. J Clin Lipidol. 2011 March-April; 5(2):105-13. doi: 10.1016/j.jacl.2011.02.001 [PMID 21392724]). Indeed, in one study of approximately 7000 participants without CV disease at baseline, LDL-attributable atherosclerotic risk was better indicated by LDL particle number when discordant with LDL-cholesterol levels (Otvos J D. et al. J Clin Lipidol. 2011 March-April; 5(2):105-13. doi: 10.1016/j.jacl.2011.02.001 [PMID 21392724]). Similarly, pathological conditions exhibiting normal LDL cholesterol levels but small and dense LDL particles (such as in familial combined hyperlipidemia, type II diabetes and abdominal obesity) (Carr M C. et al. J Clin Endocrinol Metab. 2004 June; 89(6):2601-7 [PMID 15181030]) have been found to be associated with an increased risk of myocardial infarction (Austin M A. et al. JAMA. 1988 Oct. 7; 260(13):1917-21[PMID 3418853], Stampfer M J. et al. JAMA. 1996 Sep. 18; 276(11):882-8 [PMID 8782637], Lamarche B. et al. Circulation. 1997 Jan. 7; 95(1):69-75 [PMID 8994419] and worsened CHD severity (Tornvall P. et al. Atherosclerosis. 1991 September; 90(1):67-80 [PMID 1799399], Campos H. et al. Arterioscler Thromb. 1992 February; 12(2):187-95 [PMID 1543692], Gardner C D. et al. JAMA. 1996 Sep. 18; 276(11):875-81 [PMID 8782636]). This relation may be due to the enhanced delivery of cholesterol to an atheroma by greater numbers of small and dense LDL particles (Tabas I. et al. Circulation. 2007 Oct. 16; 116(16):1832-44 [PMID 17938300]), higher susceptibility to oxidation and aggregation, reduced affinity to the LDL receptor (Berneis K K. et al. J Lipid Res. 2002 September; 43(9):1363-79 [PMID 12235168]), higher affinity to intimal proteoglycans (Anber V. et al. Arterioscler Thromb Vasc Biol. 1997 November; 17(11):2507-14 [PMID 9409221]) and higher tendency to form LDL aggregates (Hurt-Camejo E. et al. Arthritis Rheum. 2001 December; 44(12):2761-7 [PMID: 11762936]; Sartipy P. et al. J Biol Chem. 1999 Sep. 3; 274(36):25913-20 [PMID: 10464335]; Camejo G. et al. Atherosclerosis. 1998 August; 139(2):205-22 [PMID: 9712326]).

The accumulation of LDL-cholesterol in the arterial intima is a critical step in vascular cholesteryl ester (CE) deposition since increases the tendency of atherosclerotic plaque to rupture thus triggering the thrombotic process and the development of ischemic cardiomyopathy (Aikawa M. et al. Cardiovasc Pathol. 2004 May-June; 13(3):125-38 [PMID 15081469], Mauriello A. et al. Atherosclerosis. 2010 February; 208(2):572-80 [PMID 19683236], Puri R. et al. Arterioscler Thromb Vasc Biol. 2016 November; 36(11):2220-2228 [PMID 27515380]. Cholesteryl esters (CE) in the atherosclerotic plaques are deposited both extra- and intra-cellularly. Extracellular deposition of LDL-cholesteryl esters, a crucial initiating event in atherosclerosis (Skålén K. et al. Nature. 2002 Jun. 13; 417(6890):750-4 [PMID 12066187], Tabas I. et al. Circulation. 2007 Oct. 16; 116(16):1832-44 [PMID 17938300]), is mediated by the proteoglycans that conform the extracellular matrix of the arterial intima. The electrostatic interactions between proteoglycans and LDL and the proteolytic degradation of LDL, are steps extremely facilitated in the arterial intima and both promote LDL retention and aggregation (Oorni K. et al. J Lipid Res. 2000 November; 41(11):1703-14 [PMID 11060340], Oorni K. et al. Arterioscler Thromb Vasc Biol. 2005 August; 25(8):1678-83 [PMID 15879301]).

LDL aggregation has been reported to be mainly modulated by two enzymes: sphingomyelinase (SMase), secreted by endothelial cells and macrophages (Marathe S. et al. J Biol Chem. 1998 Feb. 13; 273(7):4081-8 [PMID 9461601], Schissel S L. et al. J Biol Chem. 1996 Aug. 2; 271(31):18431-6 [PMID 8702487]), and phospholipase A2 (PLA2). Both SMase and PLA2 are crucial in the process of intimal LDL aggregation during atherogenesis (Aviram M. et al. Biochem Biophys Res Commun. 1992 May 29; 185(1):465-72 [PMID 1599485], Oorni K. et al. J Biol Chem. 1998 Oct. 30; 273(44):29127-34 [PMID 9786921], Hakala J K. et al. Arterioscler Thromb Vasc Biol. 2001 June; 21(6):1053-8 [PMID 11397719], Tabas I. et al. J Biol Chem. 1993 Sep. 25; 268(27):20419-32 [PMID 8376399]). AgLDL have been detected and isolated from atherosclerotic plaques in in vivo animal models and humans (Schissel S L. et al. J Clin Invest. 1996 Sep. 15; 98(6):1455-64 [PMID 8823312], Wyler von Ballmoos M. et al. Arterioscler Thromb Vasc Biol. 2006 February; 26(2):359-64 [PMID 16322531]). Unlike native LDL (nLDL), agLDL are a potent inducer of massive intracellular CE accumulation in both macrophages (Khoo J C. et al. Arteriosclerosis. 1988 July-August; 8(4):348-58 [PMID 3395271], Zhang W Y. et al. J Biol Chem. 2000 Oct. 20; 275(42):33176-83 [PMID 10942782], Kruth H S. Curr Opin Lipidol. 2002 October; 13(5):483-8 [PMID 12352011]) and human coronary vascular smooth muscle cells (hcVSMCs) (Llorente-Cortes V. et al. Arterioscler Thromb Vasc Biol. 1998 May; 18(5): 738-46 [PMID 9598832] http://atvb.ahajournals.org/content/20/6/1572 Llorente-Cortes V. et al. Arterioscler Thromb Vasc Biol. 2002 Mar. 1; 22(3):387-93 [PMID 11884279], Llorente-Cortes V. et al. Circulation. 2002 Dec. 10; 106(24):3104-10 [PMID 12473559]). It was recently demonstrated that in hcVSMC, AgLDL are avidly taken up through the low-density lipoprotein receptor-related protein 1 (LRP1) (Llorente-Cortes V. et al. Arterioscler Thromb Vasc Biol. 1998 May; 18(5):738-46 [PMID 9598832], Llorente-Cortes V. et al. Arterioscler Thromb Vasc Biol. 2002 Mar. 1; 22(3):387-93 [PMID 11884279]. AgLDL internalization, in turn, induces LRP1 expression (Llorente-Cortes V. et al. Circulation. 2002 Dec. 10; 106(24):3104-10 [PMID 12473559], Costales P. et al. Atherosclerosis. 2010 December; 213(2):458-68 [PMID 20980003], Llorente-Cortes V. et al. J Mol Biol. 2006 Jun. 16; 359(4):950-60 [PMID 16697011]), promoting a positive feedback loop that efficiently transforms hcVSMC into foam cells. Foam cells are a crucial vascular component determining the susceptibility of atherosclerotic plaque to rupture. It has been also demonstrated that hcVSMC-foam cells synthesize and release high amounts of tissue factor, key for the prothrombotic transformation of the vascular wall and thus for the progression of atherosclerosis to thrombosis (Llorente-Cortes V. et al. Circulation. 2004 Jul. 27; 110(4):452-9 [PMID 15238452], Camino-Lopez S. et al. Cardiovasc Res. 2007 Jan. 1; 73(1):208-16 [PMID 17141748], Camino-Lopez S. et al. Thromb Haemost. 2009 December; 7(12):2137-46 [PMID 19817993]). The relevance of this mechanism in atherosclerosis is evident since VSMC are the main cellular component of the vascular wall and more than 50% of the foam cells in human atherosclerotic plaques, previously regarded as monocyte-derived macrophages, originate from VSMC (Allahverdian S. et al. Circulation. 2014 Apr. 15; 129(15):1551-9 [PMID 24481950]). Taken together, these findings support that LRP1 plays a crucial role in the generation of hVSMC-derived foam cells through LRP1-mediated AgLDL uptake and cholesterol accumulation in the vasculature susceptible to develop atherosclerosis.

LRP1 is a key signalling protein and thus is involved in various biological processes, including lipoprotein metabolism, and diseases, such as atherosclerosis (Etique N. et al. Biomed Res Int. 2013; 2013:152163 [PMID 23936774], Lillis A P. et al. J Thromb Haemost. 2005 August; 3(8):1884-93 [PMID 16102056]). AgLDL is the first ligand reported to interact with a unique LRP1 domain, CR9, which seems to exclusively recognize agLDL. AgLDL are thus specifically recognized by the region Gly1127-Cys1140 (peptide LP3: H-GDNDSEDNSDEENC-NH₂ SEQID NO: 1) that spans the C-terminal half of domain CR9, which has been reported to be crucial for binding to AgLDL and its subsequent internalization in human VSMC (Costales P. et al. J Biol Chem. 2015 Jun. 12; 290(24):14852-65 [PMID 25918169]).

Currently the prevention of atherosclerosis and other CV diseases is mainly based on lipid-lowering agents (i.e., HMG-CoA reductase and PCSK9 inhibitors) that reduce blood cholesterol levels. Although this reduction of plasma cholesterol levels undoubtedly affects the amount of cholesterol that is retained and accumulates in the vascular wall of the coronary vessels, it was found to be not enough to avoid the risk and the mortality of CV events. Indeed, the benefit of lipid-lowering drugs, such as statin-based therapies is often not related to a sharp decrease in CV mortality (acute myocardial infarction and angina pectoris) in atherosclerosis patients (DuBroff R. et al. World J Cardiol. 2015 Jul. 26; 7(7):404-9 [PMID 26225201]).

These data clearly demonstrate the crucial need to block atherosclerotic process acting on mechanisms that take place specifically in the vascular wall, by controlling the AgLDL internalization in VSMC and/or modulating the inflammatory component of the atherosclerotic plaque.

Moreover, although statin-based therapy is generally well tolerated and highly effective in lowering blood cholesterol levels, it can be associated with various adverse events (e.g, intolerance, myalgia, myopathy, rhabdomyolysis, and diabetes mellitus, among others) (Toth P P. et al. Am J Cardiovasc Drugs. 2018 June; 18(3):157-173 [PMID 29318532]). And still more, it is also demonstrated that it is generally related to an increased incidence of diabetes (Barylski M. et al. Curr Pharm Des. 2014; 20(22):3657-64 [PMID 24040871]). Indeed, being exactly diabetic patients the ones with a higher incidence of atherosclerosis and cardiovascular pathology, the development of innovative drugs for the treatment of atherosclerosis in diabetic patient or with high susceptibility of diabetes development is becoming of paramount importance.

In this context, inhibiting vascular cholesterol accumulation by modulating not only LDL aggregation but also aggregated LDL internalization by vascular cells appear to be a promising therapeutical strategy in the treatment of cardiovascular disease. Moreover, the pivotal role of the lipoprotein receptor LRP1, an in particular of the C-terminal half of CR9 domain (peptide LP3) in agLDL binding and internalization by hcVSMC, suggest that compounds derived from LP3 sequence would be highly promising compounds which would decrease LDL aggregation, agLDL internalization and VSMC-foam cell formation.

In patients with ischemic stroke, an elevation of electronegative LDL has been reported (Podrez E A. et al. Blood. 2016 Mar. 10; 127(10):1221-2. [PMID: 26965920]). Electronegative LDL is a fraction of smaller LDL with higher tendency for LDL aggregation (Bancells C. et al. J Lipid Res. 2009 March; 50(3):446-55. Bancells C. et al. Biochemistry. 2008 Aug. 5; 47(31):8186-94; Bancells C. et al. J Biol Chem. 2010 Oct. 15; 285(42):32425-35) with predictive value in cardiovascular risk (Ivanova E A et al. Vasc Health Risk Manag. 2015 Aug. 28; 11:525-32).

In peripheral artery disease (PAD), atherogenic dyslipemia, characterized by increased number of small LDL particle with higher tendency for aggregation, is the primary lipid driver for PAD risk (Aday A W. et al. Lipoprotein Particle Profiles, Standard Lipids, and Peripheral Artery Disease Incidence—Prospective Data from the Women's Health Study (Circulation. 2018, in press).

Type III hyperlipoproteinemia (HLP), or dysbetalipoproteinemia, and familial hypercholesterolemia (HF) are genetic disorders of lipid metabolism that predisposes affected subjects to the premature development of atherosclerosis. These disorders are characterized by elevated plasma cholesterol (Mahley R W et al. J Lipid Res. 1999 November; 40(11):1933-49; Santos R D et al. Lancet Diabetes Endocrinol. 2016 October; 4(10):850-61. doi: 10.1016/S2213-8587(16)30041-9. Epub 2016 May 27. Review. PMID: 27246162; Sturm A C, et al. J Am Coll Cardiol. 2018 Aug. 7; 72(6):662-680. PMID:30071997).

Familial combined hiperlipidemia (FCHL), is a common primary dyslipidemia that courses with hypercholesterolemia or high LDL cholesterol levels in which apolipoprotein B containing particle composition is abnormal and interferes with LDL-C estimation (Mehta R, et al. Atherosclerosis. 2018 October; 277:204-210. PMID: 29970255).

Type 2 diabetic (T2DM) and obese patients are characterized by atherogenic dyslipemia, characterized by increased number of small LDL particle with higher tendency for aggregation (Millán J et al. Med Clin (Barc). 2013 Nov. 16; 141(10):430-6. PMID: 23246165; Athyros V G, et al. Hormones (Athens). 2018 March; 17(1):61-67. PMID: 29858856; Iqbal J, et al. Curr Diabetes Rev. 2018; 14(5):427-433. doi: PMID:28677496; Gerber P A, Nikolic D, Rizzo M. Small, dense LDL: an update. Curr Opin Cardiol. 2017 July; 32(4):454-459. PMID: 28426445; Sartipy P, et al. J Biol Chem. 1999 Sep. 3; 274(36):25913-20. PubMed PMID: 10464335; Camejo G, et al. Atheroscler Suppl. 2002 May; 3(1):3-9. Review. PMID: 12044579). Therefore, this type of dyslipemia is likely one of the primary causes of the higher prevalence of atherosclerosis in this group of patients.

Hypothyroidism negatively affects lipid metabolism leading to hypercholesterolemia which progressively increases the risk for cardiovascular disease and, potentially, mortality. Hypercholesterolemia in hypothyroidism is mainly due to a reduction in LDL receptor activity (Jayasingh I A et al. J Family Med Prim Care. 2016 October-December; 5(4):809-816. doi: 10.4103/2249-4863.201177; Duntas L H et al, Front Endocrinol (Lausanne) 2018; 9: 511.PMCID: PMC6129606).

Cushing's syndrome (CS), including visceral obesity, dyslipidemia, hypertension and diabetes among its many manifestations, is “a model” of metabolic syndrome. Dyslipidemia is a common finding in CS as a consequence of GC-related increased lipolysis, lipogenesis and adipogenesis. Ferran F, et al. Front Horm Res. 2018; 49:85-103. doi: 10.1159/000486002. PMID: 29894989.

### BRIEF DESCRIPTION OF THE INVENTION

The inventors have successfully found that a family of compounds of formula (I) are capable of efficiently preventing LDL aggregation and VSMC-foam cell formation. These properties make the compounds of the present invention ideal candidates for the use in the therapy of atherosclerosis and all the atherosclerotic cardiovascular diseases (ASCVD) (e.g., coronary artery disease, stroke, peripheral artery disease, angina pectoris, thrombosis) as well as hypercolesterolemic conditions and/or abnormalities in lipoprotein metabolism (e.g. hyperlipidemia, hyperlipoproteinemia type II, familial hypercholesterolemia, familial combined hyperlipidemia, type II diabetes, hypothyroidism, Cushing's syndrome, obesity).

“LDL aggregation” refers to a process characterized in that Low-Density Lipoprotein (LDL) particles circulating in the blood bind to and are retained by extracellular matrix components such as proteoglycans in the arterial wall. The retained lipoproteins subsequently undergo various modifications, including oxidation, lipolysis, and proteolysis by resident hydrolytic and oxidative enzymes. These modifications cause LDL fusion that further augments LDL retention in the arterial wall, triggering a cascade of inflammatory and apoptotic responses that contribute to atherogenesis. Thus, the compounds of the invention prevent this interaction of LDL particles with the arteria wall and/or its retention.

“LDL aggregation” can be measured by LDL turbidimetry (absorbance at 405 nm) after LDL isolation from serum/plasma (standardized method to measure this process in patients has been reported in Ruuth M et al. Eur Heart Journal 2018) thus “preventing LDL aggregation” will reduce or impede the measured levels.

“VSMC-foam cell formation” refers to “Vascular smooth muscle cell (VSMC) foam cell formation”; these “VSMC-foam cells” are fat-laden vascular smooth muscle cells that have developed in response to an initial lipid injury and acquire a synthetic and proliferative phenotype and lose several markers of their physiological contractile function. Foam VSMCs can evolve either towards cell death, promoting intimal proliferation of adjacent VSMCs and plaque calcification, hence participating in atherome progression, or towards more complex and tissue-integrated responses. Lipid LDL contents, such as free cholesterol and cholesterol crystal or metabolized phospholipids can be accumulated within cells and released into the extracellular space reinforcing the lesion progression.

LDL aggregation and VSMC-foam cell formation are two events related with atherosclerosis, a disease in which the inside of an artery narrows due to the buildup of an atherome, also known as atheroatheroma or atheromatous plaque (“plaque”). Atherosclerosis is initiated by inflammatory processes in the endothelial cells of the vessel wall associated with retained low-density lipoprotein (LDL) particles. The ensuing inflammation leads to formation of atheromatous plaques in the arterial tunica intima. Macrophages recruited to the inflammation site become foam cells and platelets encourage the migration and proliferation of smooth muscle cells, which in turn ingest lipids, become replaced by collagen and transform into foam cells themselves (VSMC-foam cells). A protective fibrous cap normally forms between the fatty deposits and the artery lining (the intima), thus narrowing the lumen and stiffening the arterial wall.

Atherosclerotic cardiovascular diseases (ASCVD) are characterised by the buildup of atheromas in arteries, the list of ASCV diseases includes coronary artery disease, stroke, peripheral artery disease, angina pectoris and thrombosis. Some hypercolesterolemic conditions and/or abnormalities in lipoprotein metabolism also prone to develop these arterial atheromas such as hyperlipidemia, hyperlipoproteinemia type II, familial hypercholesterolemia, familial combined hyperlipidemia, type II diabetes, hypothyroidism, Cushing's syndrome, obesity.

Therefore, in a first aspect the invention relates to compounds having the formula (I):

R¹-AA¹-AA²-AA³-AA⁴-AA⁵-D-Glu-D-Asp-AA⁶-AA⁷-AA⁸-D-Glu-D-Glu-AA⁹-NH₂ (I)

wherein
R¹ is a C₂₋₄ acyl group,
AA¹ is either absent or is Gly
AA² is either absent or is D-Asp
AA³ is either absent or is D-Asn
AA⁴ is either absent or is D-Asp
AA⁵ is selected from the group consisting of D-Ser and D-Ala
AA⁶ is selected from the group consisting of D-Asn and D-Gln
AA⁷ is selected from the group consisting of D-Ala, D-Arg, D-Lys and D-Ser
AA⁸ is selected from the group consisting of D-Asp and D-Glu
AA⁹ is selected from the group consisting of D-Ala, D-Arg, D-Asn, D-Gln, D-Leu and D-Trp
   or a salt thereof.

In a second aspect this invention relates to processes for the preparation of a compound of formula (I) as defined in the first aspect or a pharmaceutically acceptable salt thereof.

In a third aspect this invention relates to pharmaceutical compositions comprising a compound of formula (I) as defined in the first aspect, or a pharmaceutically acceptable salt thereof and a pharmaceutically acceptable carrier, adjuvant or vehicle.

In a fourth aspect this invention relates to a compound of formula (I) as defined in the first aspect, or a pharmaceutically acceptable salt thereof or to a pharmaceutical compositions as defined in the third aspect, for use as a medicament, particularly for the prevention and/or treatment of conditions wherein decrease of vascular cholesterol accumulation, inhibition of LDL aggregation; prevention of aggregated LDL (agLDL) internalization and/or inhibition or reduction of the formation of VSMC foam cells is useful, in particular for the prevention or treatment of atherosclerosis and all the atherosclerotic cardiovascular diseases (ASCVD) (such as, coronary artery disease, stroke, peripheral artery disease, angina pectoris, thrombosis) as well as hypercolesterolemic conditions and/or abnormalities in lipoprotein metabolism (e.g. hyperlipidemia, hyperlipoproteinemia type II, familial hypercholesterolemia, familial combined hyperlipidemia, type II diabetes, hypothyroidism, Cushing's syndrome, obesity).

In a fifth aspect of this invention refers to a method for the treatment or prophylaxis of conditions wherein decrease of vascular cholesterol accumulation, inhibition of LDL aggregation; prevention of aggregated LDL (agLDL) internalization and/or inhibition or reduction of the formation of VSMC foam cells is useful, in particular for the prevention or treatment of atherosclerosis and all the atherosclerotic cardiovascular diseases (ASCVD) (e.g., coronary artery disease, stroke, peripheral artery disease, angina pectoris, thrombosis) as well as hypercolesterolemic conditions and/or abnormalities in lipoprotein metabolism (e.g. hyperlipidemia, hyperlipoproteinemia type II, familial hypercholesterolemia, familial combined hyperlipidemia, type II diabetes, hypothyroidism, Cushing's syndrome, obesity) wherein a therapeutic amount of a compound of formula (I) as defined in the first aspect, or a pharmaceutically acceptable salt, prodrug or solvate therefore, or of a pharmaceutical compositions as defined in the third aspect is administered to a patient in need of said treatment.

In a sixth aspect this invention relates to the use of a compound of formula (I) as defined in the first aspect, or a pharmaceutically acceptable salt thereof, or of a pharmaceutical compositions as defined in the third aspect for the preparation of a medicament, particularly for the prevention and/or treatment of conditions wherein decrease of vascular cholesterol accumulation, inhibition of LDL aggregation; prevention of aggregated LDL (agLDL) internalization and/or inhibition or reduction of the formation of VSMC foam cells is useful, in particular for the prevention or treatment of a disease selected from the group consisting of atherosclerosis and all the atherosclerotic cardiovascular diseases (ASCVD) (e.g., coronary artery disease, stroke, peripheral artery disease, angina pectoris, thrombosis) as well as hypercolesterolemic conditions and/or abnormalities in lipoprotein metabolism (e.g. hyperlipidemia, hyperlipoproteinemia type II, familial hypercholesterolemia, familial combined hyperlipidemia, type II diabetes, hypothyroidism, Cushing's syndrome, obesity).

These aspects and preferred embodiments thereof are additionally also defined in the claims.

### DETAILED DESCRIPTION OF THE INVENTION

In the context of the present invention some abbreviations and acronyms have been use and their meanings are provided below:
Ac Acetyl
agLDL Aggregated LDL
D-Ala D-alanine
Alloc Allyloxycarbonyl
ApoB Apolipoportein B
D-Arg D-arginine
D-Asn D-asparagine
D-Asp D-aspartic acid
ASCVD Atherosclerotic cardiovascular diseases
eq Equivalent
Boc Butyloxycarbonyl
° C. Celsius degree
CE Cholesteryl esters
CV Cardiovascular
DBU 1,8-Diazabicyclo[5.4.0]undec-7-ene
BSA Bovine serum albumin
DCM Dichloromethane
DIC N,N′-Diisopropylcarbodiimide
DIEA N,N-diisopropylethylamine
DMF Dimethylformamide
DMSO Dimethyl sulfoxide
EDTA Ethylendiaminetetraacetic acid
FC Free cholesterol
Fmoc Fluorenylmethyloxycarbonyl
D-Gln D-glutamine
D-Glu D-glutamic acid
Gly glycine
HMG-CoA β-Hydroxy β-methylglutaryl-Coenzyme A
HPLC High Performance Liquid Chromatography
HPLC-MS High Performance Liquid Chromatography coupled to mass spectometer
hVSMC Human coronary vascular smooth muscle cells
D-Leu D-leucine
LDL Low-density lipoprotein
D-Lys D-lysine
M Molar
MeOH Methanol
OtBu tert-butyl ester
Oxyma pure Ethyl cyano(hydroxyimino)acetate
Pbf 2,2,4,6,7-pentamethyldihydrobenzofuran-5-sulfonyl
PBS Phosphate buffered saline
PCSK9 Proprotein convertase subtilisin/kexin type 9
PLA2 Phospholipase A2
rpm Revolutions per minute
D-Ser D-serine
SMASE Sphingomyelinase
TFA Trifluoroacetic acid
TIS Triisopropylsilane
Tris Tris(hydroxymethyl)aminomethane
D-Trp D-tryptophan
Trt Trityl
VLDL Very low density lipoprotein

In the present invention the notation -AAⁿ- and the notation -D-xxx- wherein “xxx” stands for the 3-letters amino acid abbreviation code, are used to designate an amino acid whose amino group is devoid of one of its hydrogen atoms (H) and its carboxylic group is devoid of the rest OH. As a mere example -D-Arg- is used to represent D-Arginine whose α-amino group lacks a hydrogen and its α-carboxylic group lacks the rest OH.

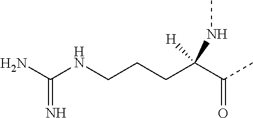

The term “salt” must be understood as any form of an active compound used in accordance with this invention in which said compound is in ionic form or is charged and coupled (associated) to a counter-ion (a cation or anion) either in solid form or in solution. This definition also includes quaternary ammonium salts. The definition includes in particular physiologically acceptable salts; this term must be understood as equivalent to “pharmacologically acceptable salts” or “pharmaceutically acceptable salts”.

The term “pharmaceutically acceptable salts” in the context of this invention means any salt that is tolerated physiologically (normally meaning that it is not toxic, particularly, as a result of the counter-ion) when used in an appropriate manner for the treatment, applied or used, particularly, in humans and/or mammals. These physiologically acceptable salts may be formed with cations associated to negatively charged groups of the peptides or anions associated with positively charged groups of the peptides. Said charged groups may be the amino terminal (—NH₂) group when R¹ is hydrogen or lateral side-chain groups such as the carboxylic lateral group of aspartic acid and glutamic acid, the amino lateral side-chain group of lysine or the guanidino lateral side-chain group of arginine. This definition specifically includes in the context of this invention a salt formed by a physiologically tolerated acid, i.e. salts of a specific active compound with physiologically tolerated organic or inorganic acids—particularly when used on humans and/or mammals.

Pharmaceutically acceptable acids include inorganic acids, such as hydrochloric, sulphuric, phosphoric, diphosphoric, hydrobromic, hydroiodic and nitrate acids, and organic acids, such as citric, maleic, malic, mandelic, ascorbic, oxalic, succinic, tartaric, acetic, methanesulfonic, ethanesulfonic, benzenesulfonic and p-toluenosulfonic acids. Pharmaceutically acceptable bases include hydroxides of alkali metals (e.g. sodium or potassium), alkaline-earth metals (for example, calcium or magnesium) and organic bases (for example, alkylamines, arylalkyilamines and heterocyclic amines).

Other preferred salts according to the invention are quaternary ammonium compounds in which an equivalent of an anion (X−) is associated with the positive charge of the N atom. X− may be an anion of diverse mineral acids such as for example, chloride, bromide, iodide, sulfate, nitrate, phosphate, or an anion of an organic acid, such as acetate, maleate, fumarate, citrate, oxalate, succinate, tartrate, malate, mandelate, trifluoracetate, methanesulfonate and p-toluenesulfonate. X− is preferably an anion selected from chloride, bromide, iodide, sulfate, nitrate, acetate, maleate, oxalate, succinate and trifluoracetate. More preferably X− is chloride, bromide, trifluoracetate or methanesulfonate.

Unless otherwise stated, the compounds of the invention are also meant to include isotopically-labelled forms i.e. compounds which differ only in the presence of one or more isotopically-enriched atoms. For example, compounds having the present structures except for the replacement of at least one hydrogen atom by a deuterium or tritium, or the replacement of at least one carbon by ¹³C- or ¹⁴C-enriched carbon, or the replacement of at least one nitrogen by ¹⁵N-enriched nitrogen are within the scope of this invention.

The compounds of formula (I), or their salts or solvates (such as hydrates) are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts, solvates or prodrugs.

As noted previously, the term “pharmaceutically acceptable salts, solvates such as hydrates, prodrugs” refers to any salt, solvate, or any other compound which, upon administration to the recipient, is capable of providing (directly or indirectly) a compound as described herein. However, it will be appreciated that non-pharmaceutically acceptable salts, solvates such as hydrates and prodrugs of the compounds of formula (I) also fall within the scope of the invention since those may be useful in the preparation of pharmaceutically acceptable salts, solvates such as hydrates and prodrugs of said compounds. The preparation of salts, solvates and prodrugs can be carried out by methods known in the art.

In one embodiment of the present invention in the compounds of formula (I) R¹ is acetyl.

In another embodiment of the present invention in the compounds of formula (I) the rest AA¹ is absent.

In another embodiment of the present invention in the compounds of formula (I) AA² is absent.

In another embodiment of the present invention in the compounds of formula (I) AA³ is absent.

In another embodiment of the present invention in the compounds of formula (I) the rest wherein -AA¹-AA²-AA³-AA⁴-AA⁵- is either absent or is selected from the group consisting of -Gly-D-Asp-D-Asn-D-Asp-D-Ser, -D-Asp-D-Asn-D-Asp-D-Ser- and -D-Asn-D-Asp-D-Ser-, -D-Asp-D-Ser and -D-Asp-D-Ala-.

In another embodiment of the present invention in the compounds of formula (I) AA⁵ is D-Ser.

In another embodiment of the present invention in the compounds of formula (I) the rest -AA⁶-AA⁷-AA⁸- is selected from the group consisting of -D-Asn-D-Ser-D-Asp-, -D-Asn-D-Ala-D-Asp-, -D-Gln-D-Ser-D-Glu-, -D-Gln-D-Ala-D-Glu-, -D-Asn-D-Arg-D-Asp- and -D-Asn-D-Lys-D-Asp-.

In still another embodiment of the present invention in the compounds of formula (I) the rest -AA⁶-AA⁷-AA⁸- is selected from the group consisting of -D-Asn-D-Ser-D-Asp-.

In another embodiment of the present invention in the compounds of formula (I) wherein -AA⁹- is selected from the group consisting of D-Ala, D-Asn, D-Gln, D-Ala, D-Arg, D-Leu and D-Trp.

In still another embodiment of the present invention in the compounds of formula (I) wherein -AA⁹- is selected from the group consisting of D-Ala and D-Asn.

Particular individual compounds of the invention falling under formula (I) include the compounds listed below:
Ac-Gly-D-Asp-D-Asn-D-Asp-D-Ser-D-Glu-D-Asp-D-Asn-D-Ser-D-Asp-D-Glu-D-Glu-D-Asn-NH₂
Ac-D-Ser-D-Glu-D-Asp-D-Asn-D-Ser-D-Asp-D-Glu-D-Glu-D-Asn-NH₂
Ac-D-Ser-D-Glu-D-Asp-D-Asn-D-Ala-D-Asp-D-Glu-D-Glu-D-Ala-NH₂
Ac-D-Ser-D-Glu-D-Asp-D-Gln-D-Ser-D-Glu-D-Glu-D-Glu-D-Gln-NH₂
Ac-D-Ser-D-Glu-D-Asp-D-Gln-D-Ala-D-Glu-D-Glu-D-Glu-D-Ala-NH₂
Ac-D-Ser-D-Glu-D-Asp-D-Asn-D-Arg-D-Asp-D-Glu-D-Glu-D-Arg-NH₂
Ac-D-Ser-D-Glu-D-Asp-D-Asn-D-Arg-D-Asp-D-Glu-D-Glu-D-Leu-NH₂
Ac-D-Ser-D-Glu-D-Asp-D-Asn-D-Arg-D-Asp-D-Glu-D-Glu-D-Trp-NH₂
Ac-D-Ser-D-Glu-D-Asp-D-Asn-D-Lys-D-Asp-D-Glu-D-Glu-D-Asn-NH₂
Ac-D-Asp-D-Asn-D-Asp-D-Ser-D-Glu-D-Asp-D-Asn-D-Ser-D-Asp-D-Glu-D-Glu-D-Asn-NH₂
Ac-D-Asn-D-Asp-D-Ser-D-Glu-D-Asp-D-Asn-D-Ser-D-Asp-D-Glu-D-Glu-D-Asn-NH₂
Ac-D-Asp-D-Ser-D-Glu-D-Asp-D-Asn-D-Ser-D-Asp-D-Glu-D-Glu-D-Asn-NH₂
Ac-D-Asp-D-Asn-D-Asp-D-Ser-D-Glu-D-Asp-D-Asn-D-Ser-D-Asp-D-Glu-D-Glu-D-Ala-NH₂
Ac-D-Asn-D-Asp-D-Ser-D-Glu-D-Asp-D-Asn-D-Ser-D-Asp-D-Glu-D-Glu-D-Ala-NH₂
Ac-D-Asp-D-Ser-D-Glu-D-Asp-D-Asn-D-Ser-D-Asp-D-Glu-D-Glu-D-Ala-NH₂
Ac-D-Asp-D-Ser-D-Glu-D-Asp-D-Asn-D-Ala-D-Asp-D-Glu-D-Glu-D-Ala-NH₂
Ac-D-Asp-D-Ala-D-Glu-D-Asp-D-Asn-D-Ala-D-Asp-D-Glu-D-Glu-D-Ala-NH₂
Ac-D-Asp-D-Ser-D-Glu-D-Asp-D-Asn-D-Lys-D-Asp-D-Glu-D-Glu-D-Asn-NH₂
Ac-D-Asp-D-Ser-D-Glu-D-Asp-D-Asn-D-Lys-D-Asp-D-Glu-D-Glu-D-Ala-NH₂
or a pharmaceutically acceptable salt thereof.

The compounds of formula (I) defined above can be obtained by available synthetic procedures as illustrated by the following general scheme:

### DETAILED DESCRIPTION OF THE ROUTE OF SYNTHESIS OF COMPOUNDS OF FORMULA (I)

A polymeric support (resin), suitable to be coupled to Fmoc-protected aminoacids (such as Nα-Fmoc-N-β-(Trt)-D-asparagine (Fmoc-D-Asn(Trt)-OH), Nα-Fmoc-D-alanine (Fmoc-D-Ala-OH), Nα-Fmoc-N-γ-(Trt)-D-glutamine (Fmoc-D-Gln(Trt)-OH), Nα-Fmoc-D-leucine (Fmoc-D-Leu-OH), Nα-Fmoc-N-in-Boc-D-tryptophan (Fmoc-D-Trp(Boc)-OH) and Nα-Fmoc-NG-(2,2,4,6,7-pentamethyldihydrobenzofuran-5-sulfonyl)-D-arginine (Fmoc-D-Arg(pbf)-OH)) through their carbonyl group and to yield upon cleavage a C-terminal amide group, such as H-Rink Amide-ChemMatrix® (I), is placed in syringe fitted with a polyethylene porous disk (reaction vessel).

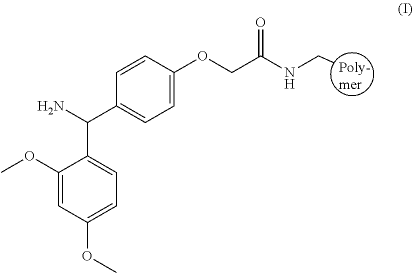

The resin is swelled by washes with appropriate organic solvents such as dichloromethane (DCM) and dimethylformamide (DMF).

Following swelling and preparation of the resin, the protected form of the first amino acid (AA⁹) (i.e. a protected amino acid selected from the group consisting of Fmoc-D-Asn(Trt)-OH, Fmoc-D-Ala-OH, Fmoc-D-Glu(Trt)-OH, Fmoc-D-Leu-OH, Fmoc-D-Trp(Boc)-OH and Fmoc-D-Arg(pbf)-OH) of the peptide to be synthetized is attached to the resin through its carboxylic acid moiety using a coupling reagent such as N,N′-Diisopropylcarbodiimide (DIC) and the use of coupling additive such as Oxyma Pure in an appropriate organic solvent such as DMF. To perform the coupling, the protected amino acid is mixed with the coupling additive and with the coupling agent. The mixture is stirred or allowed to stand for a few minutes. Then, the mixture is added to the reaction vessel containing the swelled resin and the reaction mixture is intermittently stirred for 1-5 hours. After that, the solvents and unreacted reagents are removed by suction. The performance of the reaction is monitored using a calorimetric test suitable for the detection of amines on polymeric supports, such as the Kaiser test (E. Kaiser, Anal biochem. 1970), the coupling treatment is repeated if the coupling reaction is not completed. Afterwards, the Fmoc protecting group is removed to yield product of formula (II) (wherein R_{L} represents the lateral chain of the amino acid used in the previously described coupling reaction) by treatment with an amine base solution such as a piperidine solution in DMF and/or a mixture of piperidine/DBU/toluene/DMF.

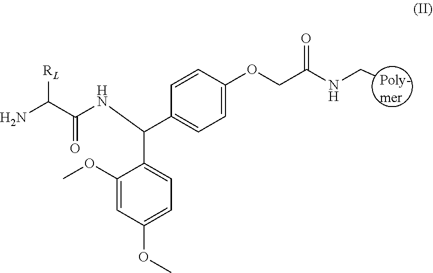

The subsequent amino acids of the peptide are coupled using the same procedure described above for the first amino acid.

Once all the amino acids have been coupled the resulting peptide (which is still bonded to the resin) is washed several times with DCM and dried by suction. Then, the peptide is cleaved from the resin using 95% TFA, 2.5% TIS and 2.5% water mixture. The acidic mixture and the resin containing the peptide are mixed and stirred at room temperature for 1-3 hours. Then, the reaction mixture is filtered and the polymeric support is rinsed with DCM. All the filtrates are pooled and the solvent is evaporated under a N₂ (flow stream) to yield the desired peptide.

### EXAMPLES

### Example 1: Ac-Gly-D-Asp-D-Asn-D-Asp-D-Ser-D-Glu-D-Asp-D-Asn-D-Ser-D-Asp-D-Glu-D-lu-D-Asn-NH₂

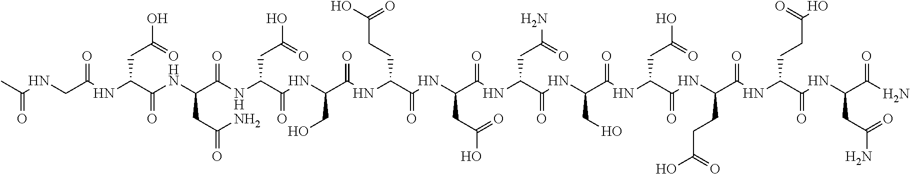 [SMILES: CC(=O)NCC(=O)N[C@H](CC(=O)O)C(=O)N[C@H](CC(N)=O)C(=O)N[C@H](CC(=O)O)C(=O)N[C@H](CO)C(=O)N[C@H](CCC(=O)O)C(=O)N[C@H](CC(=O)O)C(=O)N[C@H](CC(N)=O)C(=O)N[C@H](CO)C(=O)N[C@H](CC(=O)O)C(=O)N[C@H](CCC(=O)O)C(=O)N[C@H](CCC(=O)O)C(=O)N[C@H](CC(N)=O)C(N)=O]

Polymeric support (resin): H-Rink Amide-ChemMatrix®
Used amino acids: Fmoc-D-Asn(Trt)-OH, Fmoc-D-Glu(OtBu)-OH, Fmoc-D-Asp(OtBu)-OH, Fmoc-D-Ser(tBu)-OH and Fmoc-Gly-OH.

### General Procedure for Synthesis:

Couplings: the corresponding amino acid (3 eq), Oxyma pure (3 eq) and DIC (3 eq) are dissolved in 2 mL of DMF. Two minutes later, the reaction mixture is added to the resin. For the coupling of the first amino acid to the resin, the reaction is allowed to proceed for 3 h at room temperature with intermittent stirring. For the following amino acids the coupling time is 1 hour. Then, the reaction mixture is filtered off and the resin is washed with DMF (×5) and DCM (×5). Then, a colorimetric test is performed to check the completeness of the reaction. If the reaction is not complete, a recoupling in the same conditions is performed.

Fmoc removal is performed with treatments with a solution of 20% piperidine in DMF (2×5 min, 1×10 min). Washes with DMF (×5) and DCM (×5) are carried out after Fmoc removal. No colorimetric test is performed.

Acetylation: after last amino acid removal, the N-terminal part of the peptide is acetylated with 10 eq of DIEA and 10 eq of acetic anhydride with DCM during 15 minutes.

Cleavage: the resin is washed several times with DCM and dried. The peptide is cleaved from the resin by adding a mixture of 95% TFA, 2.5% TIS and 2.5% water at room temperature for 2 hours under intermittent stirring. The reaction mixture is filtered and rinsed with DCM (×5). All the filtrates are pooled and the solvent is evaporated using a N₂ stream. The crude of synthesis is resuspended in a mixture of water and acetonitrile (1:1), analyzed by HPLC and HPLC-MS. The crude of synthesis is purified by reverse phase chromatography, lyophilized and fully characterized.

Characterization of Example 1 after Purification:

| HPLC retention | | | Purity |
|---|---|---|---|
| time (min)¹ | Calculated MW | Observed MW² | (HPLC, Area/Area) |
| 1.55 | 1480.3 | 1502.4 [M + Na]⁺ | >99.9% |

¹HPLC: Column HPLC Sunfire C18 (4.6 mm×100 mm, 3.5 μm); flux 1 mL/min; Gradient 0-100% B in 8 min (A=0.045% TFA in H₂O and B 0.036% TFA in ACN).
²UPLC-MS: Column C18 Acquity (2.1 mm×50 mm, 1.7 μm, Waters); flux 0.6 mL/min; Gradient 0-100% B in 2 min (A=0.1% formic acid in H₂O and B 0.07% formic acid in ACN).

### Example 2: Ac-D-Ser-D-Glu-D-Asp-D-Asn-D-Ser-D-Asp-D-Glu-D-Glu-D-Asn-NH₂

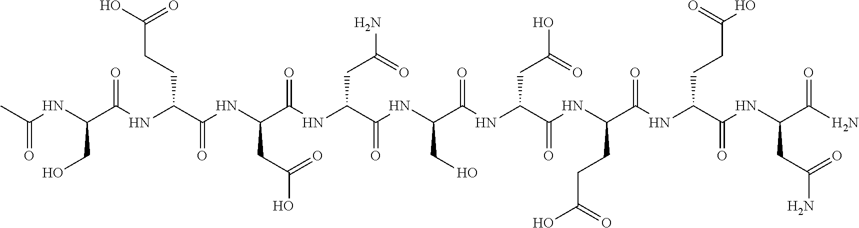 [SMILES: CC(=O)N[C@H](CO)C(=O)N[C@H](CCC(=O)O)C(=O)N[C@H](CC(=O)O)C(=O)N[C@H](CC(N)=O)C(=O)N[C@H](CO)C(=O)N[C@H](CC(=O)O)C(=O)N[C@H](CCC(=O)O)C(=O)N[C@H](CCC(=O)O)C(=O)N[C@H](CC(N)=O)C(N)=O]

Polymeric support (resin): H-Rink Amide-ChemMatrix®
Used amino acids: Fmoc-D-Asn(Trt)-OH, Fmoc-D-Glu(OtBu)-OH, Fmoc-D-Asp(OtBu)-OH and Fmoc-D-Ser(tBu)-OH.

### General Procedure for Synthesis:

Couplings: the corresponding amino acid (3 eq), Oxyma pure (3 eq) and DIC (3 eq) are dissolved in 2 mL of DMF. Two minutes later, the reaction mixture is added to the resin. For the coupling of the first amino acid to the resin, the reaction is allowed to proceed for 3 h at room temperature with intermittent stirring. For the following amino acids the coupling time is 1 hour. Then, the reaction mixture is filtered off and the resin is washed with DMF (×5) and DCM (×5). Then, a colorimetric test is performed to check the completeness of the reaction. If the reaction is not complete, a recoupling in the same conditions is performed.

Fmoc removal is performed with treatments with a solution of 20% piperidine in DMF (2×5 min, 1×10 min). Washes with DMF (×5) and DCM (×5) are carried out after Fmoc removal. No colorimetric test is performed.

Acetylation: after last amino acid removal, the N-terminal part of the peptide is acetylated with 10 eq of DIEA and 10 eq of acetic anhydride with DCM during 15 minutes.

Cleavage: the resin is washed several times with DCM and dried. The peptide is cleaved from the resin by adding a mixture of 95% TFA, 2.5% TIS and 2.5% water at room temperature for 2 hours under intermittent stirring. The reaction mixture is filtered and rinsed with DCM (×5). All the filtrates are pooled and the solvent is evaporated using a N₂ stream. The crude of synthesis is resuspended in a mixture of water and acetonitrile (1:1), analyzed by HPLC and HPLC-MS. The crude of synthesis is purified by reverse phase chromatography, lyophilized and fully characterized.

Characterization of Example 2 after Purification:

| HPLC retention | | | Purity |
|---|---|---|---|
| time (min)¹ | Calculated MW | Observed MW² | (HPLC, Area/Area) |
| 3.15 | 1078.9 | 1079.2 [M + H]⁺ | >90% |

¹HPLC: Column HPLC Sunfire C18 (4.6 mm×100 mm, 3.5 μm); flux 1 mL/min; Gradient 0-100% B in 8 min (A=0.045% TFA in H₂O and B 0.036% TFA in ACN).
²UPLC-MS: Column C18 Acquity (2.1 mm×50 mm, 1.7 μm, Waters); flux 0.6 mL/min; Gradient 0-100% B in 2 min (A=0.1% formic acid in H₂O and B 0.07% formic acid in ACN).

### Example 3: Ac-D-Ser-D-Glu-D-Asp-D-Asn-D-Ala-D-Asp-D-Glu-D-Glu-D-Ala-NH₂

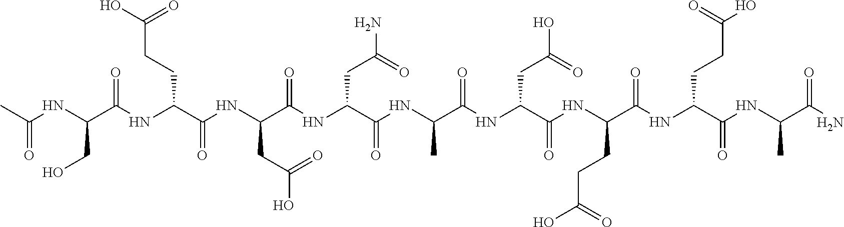 [SMILES: CC(=O)N[C@H](CO)C(=O)N[C@H](CCC(=O)O)C(=O)N[C@H](CC(=O)O)C(=O)N[C@H](CC(N)=O)C(=O)N[C@H](C)C(=O)N[C@H](CC(=O)O)C(=O)N[C@H](CCC(=O)O)C(=O)N[C@H](CCC(=O)O)C(=O)N[C@H](C)C(N)=O]

Polymeric support (resin): H-Rink Amide-ChemMatrix®
Used amino acids: Fmoc-D-Ala-OH, Fmoc-D-Asn(Trt)-OH, Fmoc-D-Glu(OtBu)-OH, Fmoc-D-Asp(OtBu)-OH and Fmoc-D-Ser(tBu)-OH.

### General Procedure for Synthesis:

Couplings: the corresponding amino acid (3 eq), Oxyma pure (3 eq) and DIC (3 eq) are dissolved in 2 mL of DMF. Two minutes later, the reaction mixture is added to the resin. For the coupling of the first amino acid to the resin, the reaction is allowed to proceed for 3 h at room temperature with intermittent stirring. For the following amino acids the coupling time is 1 hour. Then, the reaction mixture is filtered off and the resin is washed with DMF (×5) and DCM (×5). Then, a colorimetric test is performed to check the completeness of the reaction. If the reaction is not complete, a recoupling in the same conditions is performed.

Fmoc removal is performed with treatments with a solution of 20% piperidine in DMF (2×5 min, 1×10 min). Washes with DMF (×5) and DCM (×5) are carried out after Fmoc removal. No colorimetric test is performed.

Acetylation: after last amino acid removal, the N-terminal part of the peptide is acetylated with 10 eq of DIEA and 10 eq of acetic anhydride with DCM during 15 minutes.

Cleavage: the resin is washed several times with DCM and dried. The peptide is cleaved from the resin by adding a mixture of 95% TFA, 2.5% TIS and 2.5% water at room temperature for 2 hours under intermittent stirring. The reaction mixture is filtered and rinsed with DCM (×5). All the filtrates are pooled and the solvent is evaporated using a N₂ stream. The crude of synthesis is resuspended in a mixture of water and acetonitrile (1:1), analyzed by HPLC and HPLC-MS. The crude of synthesis is purified by reverse phase chromatography, lyophilized and fully characterized.

Characterization of Example 3 after Purification:

| HPLC retention | | | Purity |
|---|---|---|---|
| time (min)¹ | Calculated MW | Observed MW² | (HPLC, Area/Area) |
| 3.34 | 1019.9 | 1020.3 [M + H]⁺ | >97% |

¹HPLC: Column HPLC Sunfire C18 (4.6 mm×100 mm, 3.5 μm); flux 1 mL/min; Gradient 0-100% B in 8 min (A=0.045% TFA in H₂O and B 0.036% TFA in ACN).
²UPLC-MS: Column C18 Acquity (2.1 mm×50 mm, 1.7 μm, Waters); flux 0.6 mL/min; Gradient 0-100% B in 2 min (A=0.1% formic acid in H₂O and B 0.07% formic acid in ACN).

### Example 4: Ac-D-Ser-D-Glu-D-Asp-D-Gln-D-Ser-D-Glu-D-Glu-D-Glu-D-Gln-NH₂

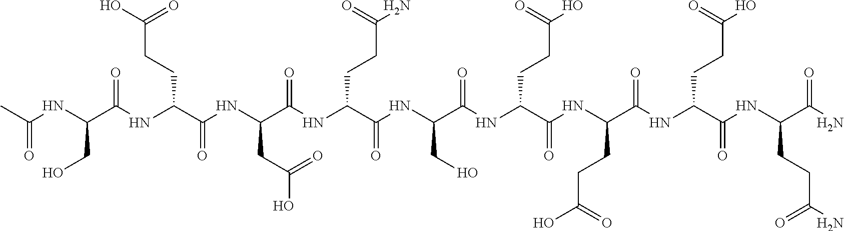 [SMILES: CC(=O)N[C@H](CO)C(=O)N[C@H](CCC(=O)O)C(=O)N[C@H](CC(=O)O)C(=O)N[C@H](CCC(N)=O)C(=O)N[C@H](CO)C(=O)N[C@H](CCC(=O)O)C(=O)N[C@H](CCC(=O)O)C(=O)N[C@H](CCC(=O)O)C(=O)N[C@H](CCC(N)=O)C(N)=O]

Polymeric support (resin): H-Rink Amide-ChemMatrix®
Used amino acids: Fmoc-D-Gln(Trt)-OH, Fmoc-D-Glu(OtBu)-OH, Fmoc-D-Asp(OtBu)-OH and Fmoc-D-Ser(tBu)-OH.

### General Procedure for Synthesis:

Couplings: the corresponding amino acid (3 eq), Oxyma pure (3 eq) and DIC (3 eq) are dissolved in 2 mL of DMF. Two minutes later, the reaction mixture is added to the resin. For the coupling of the first amino acid to the resin, the reaction is allowed to proceed for 3 h at room temperature with intermittent stirring. For the following amino acids the coupling time is 1 hour. Then, the reaction mixture is filtered off and the resin is washed with DMF (×5) and DCM (×5). Then, a colorimetric test is performed to check the completeness of the reaction. If the reaction is not complete, a recoupling in the same conditions is performed.

Fmoc removal is performed with treatments with a solution of 20% piperidine in DMF (2×5 min, 1×10 min). Washes with DMF (×5) and DCM (×5) are carried out after Fmoc removal. No colorimetric test is performed.

Acetylation: after last amino acid removal, the N-terminal part of the peptide is acetylated with 10 eq of DIEA and 10 eq of acetic anhydride with DCM during 15 minutes.

Cleavage: the resin is washed several times with DCM and dried. The peptide is cleaved from the resin by adding a mixture of 95% TFA, 2.5% TIS and 2.5% water at room temperature for 2 hours under intermittent stirring. The reaction mixture is filtered and rinsed with DCM (×5). All the filtrates are pooled and the solvent is evaporated using a N₂ stream. The crude of synthesis is resuspended in a mixture of water and acetonitrile (1:1), analyzed by HPLC and HPLC-MS. The crude of synthesis is purified by reverse phase chromatography, lyophilized and fully characterized.

Characterization of Example 4 after Purification:

| HPLC retention | | | Purity |
|---|---|---|---|
| time (min)¹ | Calculated MW | Observed MW² | (HPLC, Area/Area) |
| 3.24 | 1121.0 | 1121.5 [M + H]⁺ | >91% |

¹HPLC: Column HPLC Sunfire C18 (4.6 mm×100 mm, 3.5 μm); flux 1 mL/min; Gradient 0-100% B in 8 min (A=0.045% TFA in H₂O and B 0.036% TFA in ACN).
²UPLC-MS: Column C18 Acquity (2.1 mm×50 mm, 1.7 μm, Waters); flux 0.6 mL/min; Gradient 0-100% B in 2 min (A=0.1% formic acid in H₂O and B 0.07% formic acid in ACN).

### Example 5: Ac-D-Ser-D-Glu-D-Asp-D-Gln-D-Ala-D-Glu-D-Glu-D-Glu-D-Ala-NH₂

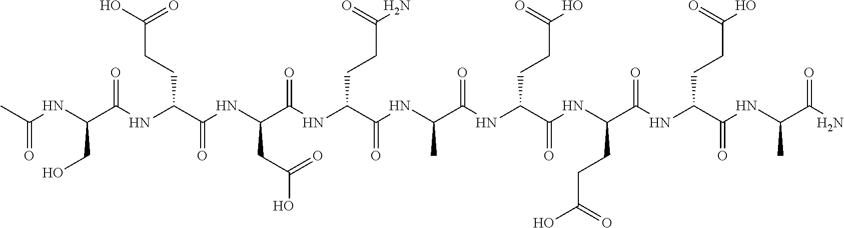 [SMILES: CC(=O)N[C@H](CO)C(=O)N[C@H](CCC(=O)O)C(=O)N[C@H](CC(=O)O)C(=O)N[C@H](CCC(N)=O)C(=O)N[C@H](C)C(=O)N[C@H](CCC(=O)O)C(=O)N[C@H](CCC(=O)O)C(=O)N[C@H](CCC(=O)O)C(=O)N[C@H](C)C(N)=O]

Polymeric support (resin): H-Rink Amide-ChemMatrix®
Used amino acids: Fmoc-D-Ala-OH. Fmoc-D-Gln(Trt)-OH, Fmoc-D-Glu(OtBu)-OH, Fmoc-D-Asp(OtBu)-OH and Fmoc-D-Ser(tBu)-OH.

### General Procedure for Synthesis:

Couplings: the corresponding amino acid (3 eq), Oxyma pure (3 eq) and DIC (3 eq) are dissolved in 2 mL of DMF. Two minutes later, the reaction mixture is added to the resin. For the coupling of the first amino acid to the resin, the reaction is allowed to proceed for 3 h at room temperature with intermittent stirring. For the following amino acids the coupling time is 1 hour. Then, the reaction mixture is filtered off and the resin is washed with DMF (×5) and DCM (×5). Then, a colorimetric test is performed to check the completeness of the reaction. If the reaction is not complete, a recoupling in the same conditions is performed.

Fmoc removal is performed with treatments with a solution of 20% piperidine in DMF (2×5 min, 1×10 min). Washes with DMF (×5) and DCM (×5) are carried out after Fmoc removal. No colorimetric test is performed.

Acetylation: after last amino acid removal, the N-terminal part of the peptide is acetylated with 10 eq of DIEA and 10 eq of acetic anhydride with DCM during 15 minutes.

Cleavage: the resin is washed several times with DCM and dried. The peptide is cleaved from the resin by adding a mixture of 95% TFA, 2.5% TIS and 2.5% water at room temperature for 2 hours under intermittent stirring. The reaction mixture is filtered and rinsed with DCM (×5). All the filtrates are pooled and the solvent is evaporated using a N₂ stream. The crude of synthesis is resuspended in a mixture of water and acetonitrile (1:1), analyzed by HPLC and HPLC-MS. The crude of synthesis is purified by reverse phase chromatography, lyophilized and fully characterized.

Characterization of Example 5 after Purification:

| HPLC retention | | | Purity |
|---|---|---|---|
| time (min)¹ | Calculated MW | Observed MW² | (HPLC, Area/Area) |
| 3.47 | 1047.9 | 1048.2 [M + H]⁺ | >92% |

¹HPLC: Column HPLC Sunfire C18 (4.6 mm×100 mm, 3.5 μm); flux 1 mL/min; Gradient 0-100% B in 8 min (A=0.045% TFA in H₂O and B 0.036% TFA in ACN).
²UPLC-MS: Column C18 Acquity (2.1 mm×50 mm, 1.7 μm, Waters); flux 0.6 mL/min; Gradient 0-100% B in 2 min (A=0.1% formic acid in H₂O and B 0.07% formic acid in ACN).

### Example 6: Ac-D-Ser-D-Glu-D-Asp-D-Asn-D-Arg-D-Asp-D-Glu-D-Glu-D-Arg-NH₂

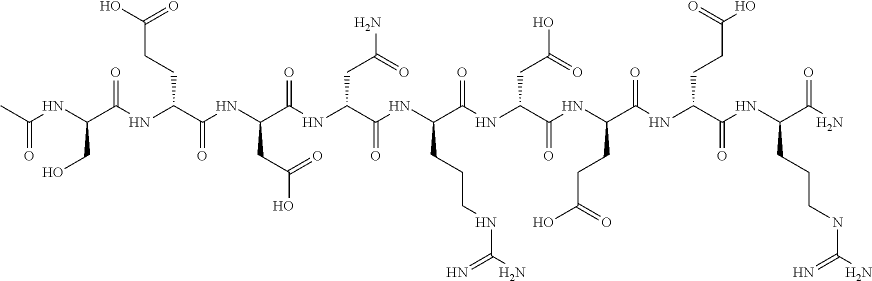 [SMILES: CC(=O)N[C@H](CO)C(=O)N[C@H](CCC(=O)O)C(=O)N[C@H](CC(=O)O)C(=O)N[C@H](CC(N)=O)C(=O)N[C@H](CCCNC(=N)N)C(=O)N[C@H](CC(=O)O)C(=O)N[C@H](CCC(=O)O)C(=O)N[C@H](CCC(=O)O)C(=O)N[C@H](CCCNC(=N)N)C(N)=O]

Polymeric support (resin): H-Rink Amide-ChemMatrix®
Used amino acids: Fmoc-D-Arg(pbf)-OH, Fmoc-D-Asn(Trt)-OH, Fmoc-D-Glu(OtBu)-OH, Fmoc-D-Asp(OtBu)-OH and Fmoc-D-Ser(tBu)-OH.

### General Procedure for Synthesis:

Couplings: the corresponding amino acid (3 eq), Oxyma pure (3 eq) and DIC (3 eq) are dissolved in 2 mL of DMF. Two minutes later, the reaction mixture is added to the resin. For the coupling of the first amino acid to the resin, the reaction is allowed to proceed for 3 h at room temperature with intermittent stirring. For the following amino acids the coupling time is 1 hour. Then, the reaction mixture is filtered off and the resin is washed with DMF (×5) and DCM (×5). Then, a colorimetric test is performed to check the completeness of the reaction. If the reaction is not complete, a recoupling in the same conditions is performed.

Fmoc removal is performed with treatments with a solution of 20% piperidine in DMF (2×5 min, 1×10 min). Washes with DMF (×5) and DCM (×5) are carried out after Fmoc removal. No colorimetric test is performed.

Acetylation: after last amino acid removal, the N-terminal part of the peptide is acetylated with 10 eq of DIEA and 10 eq of acetic anhydride with DCM during 15 minutes.

Cleavage: the resin is washed several times with DCM and dried. The peptide is cleaved from the resin by adding a mixture of 95% TFA, 2.5% TIS and 2.5% water at room temperature for 2 hours under intermittent stirring. The reaction mixture is filtered and rinsed with DCM (×5). All the filtrates are pooled and the solvent is evaporated using a N₂ stream. The crude of synthesis is resuspended in a mixture of water and acetonitrile (1:1), analyzed by HPLC and HPLC-MS. The crude of synthesis is purified by reverse phase chromatography, lyophilized and fully characterized.

Characterization of Example 6 after Purification:

| HPLC retention | | | Purity |
|---|---|---|---|
| time (min)¹ | Calculated MW | Observed MW² | (HPLC, Area/Area) |
| 3.10 | 1190.1 | 1190.5 [M + H]⁺ | >94% |

¹HPLC: Column HPLC Sunfire C18 (4.6 mm×100 mm, 3.5 μm); flux 1 mL/min; Gradient 0-100% B in 8 min (A=0.045% TFA in H₂O and B 0.036% TFA in ACN).
²UPLC-MS: Column C18 Acquity (2.1 mm×50 mm, 1.7 μm, Waters); flux 0.6 mL/min; Gradient 0-100% B in 2 min (A=0.1% formic acid in H₂O and B 0.07% formic acid in ACN).

### Example 7: Ac-D-Ser-D-Glu-D-Asp-D-Asn-D-Arg-D-Asp-D-Glu-D-Glu-D-Leu-NH₂

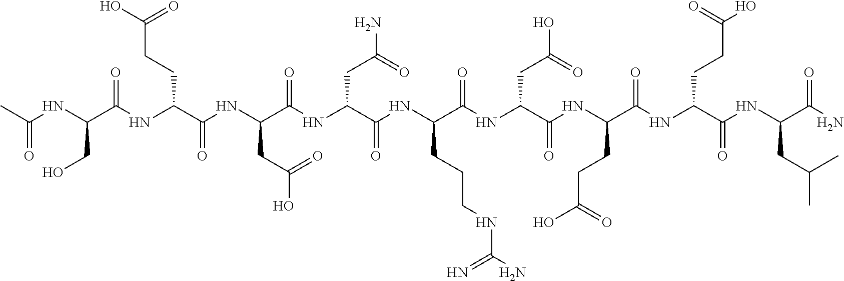 [SMILES: CC(=O)N[C@H](CO)C(=O)N[C@H](CCC(=O)O)C(=O)N[C@H](CC(=O)O)C(=O)N[C@H](CC(N)=O)C(=O)N[C@H](CCCNC(=N)N)C(=O)N[C@H](CC(=O)O)C(=O)N[C@H](CCC(=O)O)C(=O)N[C@H](CCC(=O)O)C(=O)N[C@H](CC(C)C)C(N)=O]

Polymeric support (resin): H-Rink Amide-ChemMatrix®
Used amino acids: Fmoc-D-Leu-OH, Fmoc-D-Arg(pbf)-OH, Fmoc-D-Asn(Trt)-OH, Fmoc-D-Glu(OtBu)-OH, Fmoc-D-Asp(OtBu)-OH, and Fmoc-D-Ser(tBu)-OH.

### General Procedure for Synthesis:

Couplings: the corresponding amino acid (3 eq), Oxyma pure (3 eq) and DIC (3 eq) are dissolved in 2 mL of DMF. Two minutes later, the reaction mixture is added to the resin. For the coupling of the first amino acid to the resin, the reaction is allowed to proceed for 3 h at room temperature with intermittent stirring. For the following amino acids the coupling time is 1 hour. Then, the reaction mixture is filtered off and the resin is washed with DMF (×5) and DCM (×5). Then, a colorimetric test is performed to check the completeness of the reaction. If the reaction is not complete, a recoupling in the same conditions is performed.

Fmoc removal is performed with treatments with a solution of 20% piperidine in DMF (2×5 min, 1×10 min). Washes with DMF (×5) and DCM (×5) are carried out after Fmoc removal. No colorimetric test is performed.

Acetylation: after last amino acid removal, the N-terminal part of the peptide is acetylated with 10 eq of DIEA and 10 eq of acetic anhydride with DCM during 15 minutes.

Cleavage: the resin is washed several times with DCM and dried. The peptide is cleaved from the resin by adding a mixture of 95% TFA, 2.5% TIS and 2.5% water at room temperature for 2 hours under intermittent stirring. The reaction mixture is filtered and rinsed with DCM (×5). All the filtrates are pooled and the solvent is evaporated using a N₂ stream. The crude of synthesis is resuspended in a mixture of water and acetonitrile (1:1), analyzed by HPLC and HPLC-MS. The crude of synthesis is purified by reverse phase chromatography, lyophilized and fully characterized.

Characterization of Example 7 after Purification:

| HPLC retention | | | Purity |
|---|---|---|---|
| time (min)¹ | Calculated MW | Observed MW² | (HPLC, Area/Area) |
| 3.58 | 1147.1 | 1147.0 | >98% |

¹HPLC: Column HPLC Sunfire C18 (4.6 mm×100 mm, 3.5 μm); flux 1 mL/min; Gradient 0-100% B in 8 min (A=0.045% TFA in H₂O and B 0.036% TFA in ACN).
²UPLC-MS: Column C18 Acquity (2.1 mm×50 mm, 1.7 μm, Waters); flux 0.6 mL/min; Gradient 0-100% B in 2 min (A=0.1% formic acid in H₂O and B 0.07% formic acid in ACN).

### Example 8: Ac-D-Ser-D-Glu-D-Asp-D-Asn-D-Arg-D-Asp-D-Glu-D-Glu-D-Trp-NH₂

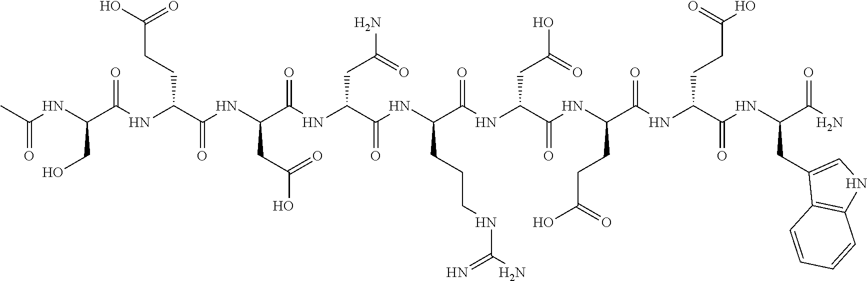 [SMILES: CC(=O)N[C@H](CO)C(=O)N[C@H](CCC(=O)O)C(=O)N[C@H](CC(=O)O)C(=O)N[C@H](CC(N)=O)C(=O)N[C@H](CCCNC(=N)N)C(=O)N[C@H](CC(=O)O)C(=O)N[C@H](CCC(=O)O)C(=O)N[C@H](CCC(=O)O)C(=O)N[C@H](Cc1c[nH]c2ccccc12)C(N)=O]

Polymeric support (resin): H-Rink Amide-ChemMatrix®
Used amino acids: Fmoc-D-Trp(Boc)-OH, Fmoc-D-Arg(pbf)-OH, Fmoc-D-Asn(Trt)-OH, Fmoc-D-Glu(OtBu)-OH, Fmoc-D-Asp(OtBu)-OH, and Fmoc-D-Ser(tBu)-OH.

### General Procedure for Synthesis:

Couplings: the corresponding amino acid (3 eq), Oxyma pure (3 eq) and DIC (3 eq) are dissolved in 2 mL of DMF. Two minutes later, the reaction mixture is added to the resin. For the coupling of the first amino acid to the resin, the reaction is allowed to proceed for 3 h at room temperature with intermittent stirring. For the following amino acids the coupling time is 1 hour. Then, the reaction mixture is filtered off and the resin is washed with DMF (×5) and DCM (×5). Then, a colorimetric test is performed to check the completeness of the reaction. If the reaction is not complete, a recoupling in the same conditions is performed.

Fmoc removal is performed with treatments with a solution of 20% piperidine in DMF (2×5 min, 1×10 min). Washes with DMF (×5) and DCM (×5) are carried out after Fmoc removal. No colorimetric test is performed.

Acetylation: after last amino acid removal, the N-terminal part of the peptide is acetylated with 10 eq of DIEA and 10 eq of acetic anhydride with DCM during 15 minutes.

Cleavage: the resin is washed several times with DCM and dried. The peptide is cleaved from the resin by adding a mixture of 95% TFA, 2.5% TIS and 2.5% water at room temperature for 2 hours under intermittent stirring. The reaction mixture is filtered and rinsed with DCM (×5). All the filtrates are pooled and the solvent is evaporated using a N₂ stream. The crude of synthesis is resuspended in a mixture of water and acetonitrile (1:1), analyzed by HPLC and HPLC-MS. The crude of synthesis is purified by reverse phase chromatography, lyophilized and fully characterized.

Characterization of Example 8 after Purification:

| HPLC retention | | | Purity |
|---|---|---|---|
| time (min)¹ | Calculated MW | Observed MW² | (HPLC, Area/Area) |
| 3.93 | 1120.1 | 1120.4 [M + H]⁺ | >96% |

¹HPLC: Column HPLC Sunfire C18 (4.6 mm×100 mm, 3.5 μm); flux 1 mL/min; Gradient 0-100% B in 8 min (A=0.045% TFA in H₂O and B 0.036% TFA in ACN).
²UPLC-MS: Column C18 Acquity (2.1 mm×50 mm, 1.7 μm, Waters); flux 0.6 mL/min; Gradient 0-100% B in 2 min (A=0.1% formic acid in H₂O and B 0.07% formic acid in ACN).

### Example 9: Ac-D-Ser-D-Glu-D-Asp-D-Asn-D-Lys-D-Asp-D-Glu-D-Glu-D-Asn-NH₂

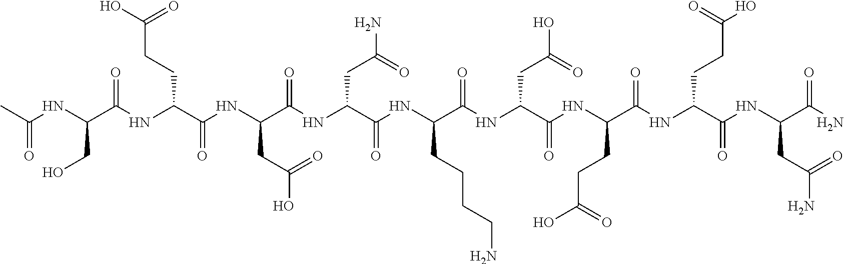 [SMILES: CC(=O)N[C@H](CO)C(=O)N[C@H](CCC(=O)O)C(=O)N[C@H](CC(=O)O)C(=O)N[C@H](CC(N)=O)C(=O)N[C@H](CCCCN)C(=O)N[C@H](CC(=O)O)C(=O)N[C@H](CCC(=O)O)C(=O)N[C@H](CCC(=O)O)C(=O)N[C@H](CC(N)=O)C(N)=O]

Polymeric support (resin): H-Rink Amide-ChemMatrix®
Used amino acids: Fmoc-D-Asn(Trt)-OH, Fmoc-D-Glu(OtBu)-OH, Fmoc-D-Asp(OtBu)-OH, Fmoc-D-Lys(Boc)-OH and Fmoc-D-Ser(tBu)-OH.

### General Procedure for Synthesis:

Couplings: the corresponding amino acid (3 eq), Oxyma pure (3 eq) and DIC (3 eq) are dissolved in 2 mL of DMF. Two minutes later, the reaction mixture is added to the resin. For the coupling of the first amino acid to the resin, the reaction is allowed to proceed for 3 h at room temperature with intermittent stirring. For the following amino acids the coupling time is 1 hour. Then, the reaction mixture is filtered off and the resin is washed with DMF (×5) and DCM (×5). Then, a colorimetric test is performed to check the completeness of the reaction. If the reaction is not complete, a recoupling in the same conditions is performed.

Fmoc removal is performed with treatments with a solution of 20% piperidine in DMF (2×5 min, 1×10 min). Washes with DMF (×5) and DCM (×5) are carried out after Fmoc removal. No colorimetric test is performed.

Acetylation: after last amino acid removal, the N-terminal part of the peptide is acetylated with 10 eq of DIEA and 10 eq of acetic anhydride with DCM during 15 minutes.

Cleavage: the resin is washed several times with DCM and dried. The peptide is cleaved from the resin by adding a mixture of 95% TFA, 2.5% TIS and 2.5% water at room temperature for 2 hours under intermittent stirring. The reaction mixture is filtered and rinsed with DCM (×5). All the filtrates are pooled and the solvent is evaporated using a N₂ stream. The crude of synthesis is resuspended in a mixture of water and acetonitrile (1:1), analyzed by HPLC and HPLC-MS. The crude of synthesis is purified by reverse phase chromatography, lyophilized and fully characterized.

Characterization of Example 9 after Purification:

| HPLC retention | | | Purity |
|---|---|---|---|
| time (min)¹ | Calculated MW | Observed MW² | (HPLC, Area/Area) |
| 3.05 | 1120.0 | 1120.2 [M + H]⁺ | >93% |

¹HPLC: Column HPLC Sunfire C18 (4.6 mm×100 mm, 3.5 μm); flux 1 mL/min; Gradient 0-100% B in 8 min (A=0.045% TFA in H₂O and B 0.036% TFA in ACN).
²UPLC-MS: Column C18 Acquity (2.1 mm×50 mm, 1.7 μm, Waters); flux 0.6 mL/min; Gradient 0-100% B in 2 min (A=0.1% formic acid in H₂O and B 0.07% formic acid in ACN).

### Example 10: Ac-D-Asp-D-Asn-D-Asp-D-Ser-D-Glu-D-Asp-D-Asn-D-Ser-D-Asp-D-Glu-D-Glu-D-Asn-NH₂

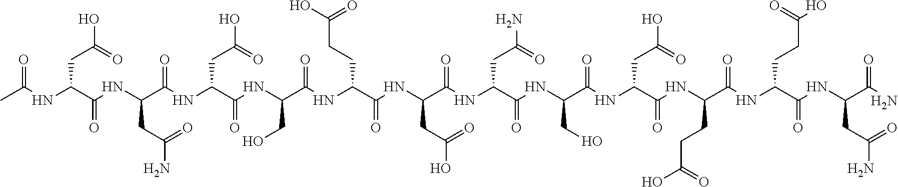 [SMILES: CC(=O)N[C@H](CC(=O)O)C(=O)N[C@H](CC(N)=O)C(=O)N[C@H](CC(=O)O)C(=O)N[C@H](CO)C(=O)N[C@H](CCC(=O)O)C(=O)N[C@H](CC(=O)O)C(=O)N[C@H](CC(N)=O)C(=O)N[C@H](CO)C(=O)N[C@H](CC(=O)O)C(=O)N[C@H](CCC(=O)O)C(=O)N[C@H](CCC(=O)O)C(=O)N[C@H](CC(N)=O)C(N)=O]

Polymeric support (resin): H-Rink Amide-ChemMatrix®
Used amino acids: Fmoc-D-Asn(Trt)-OH, Fmoc-D-Glu(OtBu)-OH, Fmoc-D-Asp(OtBu)-OH and Fmoc-D-Ser(tBu)-OH.

### General Procedure for Synthesis:

Couplings: the corresponding amino acid (3 eq), Oxyma pure (3 eq) and DIC (3 eq) are dissolved in 2 mL of DMF. Two minutes later, the reaction mixture is added to the resin. For the coupling of the first amino acid to the resin, the reaction is allowed to proceed for 3 h at room temperature with intermittent stirring. For the following amino acids the coupling time is 1 hour. Then, the reaction mixture is filtered off and the resin is washed with DMF (×5) and DCM (×5). Then, a colorimetric test is performed to check the completeness of the reaction. If the reaction is not complete, a recoupling in the same conditions is performed.

Fmoc removal is performed with treatments with a solution of 20% piperidine in DMF (2×5 min, 1×10 min). Washes with DMF (×5) and DCM (×5) are carried out after Fmoc removal. No colorimetric test is performed.

Acetylation: after last amino acid removal, the N-terminal part of the peptide is acetylated with 10 eq of DIEA and 10 eq of acetic anhydride with DCM during 15 minutes.

Cleavage: the resin is washed several times with DCM and dried. The peptide is cleaved from the resin by adding a mixture of 95% TFA, 2.5% TIS and 2.5% water at room temperature for 2 hours under intermittent stirring. The reaction mixture is filtered and rinsed with DCM (×5). All the filtrates are pooled and the solvent is evaporated using a N₂ stream. The crude of synthesis is resuspended in a mixture of water and acetonitrile (1:1), analyzed by HPLC and HPLC-MS. The crude of synthesis is purified by reverse phase chromatography, lyophilized and fully characterized.

Characterization of Example 10 after Purification:

| HPLC retention | | | Purity |
|---|---|---|---|
| time (min)¹ | Calculated MW | Observed MW² | (HPLC, Area/Area) |
| 1.35 | 1423.2 | 1424.0 [M + H]⁺ | >99% |

¹HPLC: Column HPLC Sunfire C18 (4.6 mm×100 mm, 3.5 μm); flux 1 mL/min; Gradient 0-100% B in 8 min (A=0.045% TFA in H₂O and B 0.036% TFA in ACN).
²UPLC-MS: Column C18 Acquity (2.1 mm×50 mm, 1.7 μm, Waters); flux 0.6 mL/min; Gradient 0-100% B in 2 min (A=0.1% formic acid in H₂O and B 0.07% formic acid in ACN).

### Example 11: Ac-D-Asn-D-Asp-D-Ser-D-Glu-D-Asp-D-Asn-D-Ser-D-Asp-D-Glu-D-Glu-D-Asn-NH₂

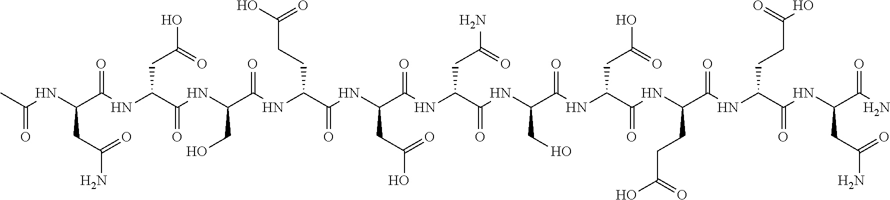 [SMILES: CC(=O)N[C@H](CC(N)=O)C(=O)N[C@H](CC(=O)O)C(=O)N[C@H](CO)C(=O)N[C@H](CCC(=O)O)C(=O)N[C@H](CC(=O)O)C(=O)N[C@H](CC(N)=O)C(=O)N[C@H](CO)C(=O)N[C@H](CC(=O)O)C(=O)N[C@H](CCC(=O)O)C(=O)N[C@H](CCC(=O)O)C(=O)N[C@H](CC(N)=O)C(N)=O]

Polymeric support (resin): H-Rink Amide-ChemMatrix®
Used amino acids: Fmoc-D-Asn(Trt)-OH, Fmoc-D-Glu(OtBu)-OH, Fmoc-D-Asp(OtBu)-OH and Fmoc-D-Ser(tBu)-OH.

### General Procedure for Synthesis:

Couplings: the corresponding amino acid (3 eq), Oxyma pure (3 eq) and DIC (3 eq) are dissolved in 2 mL of DMF. Two minutes later, the reaction mixture is added to the resin. For the coupling of the first amino acid to the resin, the reaction is allowed to proceed for 3 h at room temperature with intermittent stirring. For the following amino acids the coupling time is 1 hour. Then, the reaction mixture is filtered off and the resin is washed with DMF (×5) and DCM (×5). Then, a colorimetric test is performed to check the completeness of the reaction. If the reaction is not complete, a recoupling in the same conditions is performed.

Fmoc removal is performed with treatments with a solution of 20% piperidine in DMF (2×5 min, 1×10 min). Washes with DMF (×5) and DCM (×5) are carried out after Fmoc removal. No colorimetric test is performed.

Acetylation: after last amino acid removal, the N-terminal part of the peptide is acetylated with 10 eq of DIEA and 10 eq of acetic anhydride with DCM during 15 minutes.

Cleavage: the resin is washed several times with DCM and dried. The peptide is cleaved from the resin by adding a mixture of 95% TFA, 2.5% TIS and 2.5% water at room temperature for 2 hours under intermittent stirring. The reaction mixture is filtered and rinsed with DCM (×5). All the filtrates are pooled and the solvent is evaporated using a N₂ stream. The crude of synthesis is resuspended in a mixture of water and acetonitrile (1:1), analyzed by HPLC and HPLC-MS. The crude of synthesis is purified by reverse phase chromatography, lyophilized and fully characterized.

Characterization of Example 11 after Purification:

| HPLC retention | | | Purity |
|---|---|---|---|
| time (min)¹ | Calculated MW | Observed MW² | (HPLC, Area/Area) |
| 3.16 | 1308.1 | 1308.7 [M + H]⁺ | >91% |

¹HPLC: Column HPLC Sunfire C18 (4.6 mm×100 mm, 3.5 μm); flux 1 mL/min; Gradient 0-100% B in 8 min (A=0.045% TFA in H₂O and B 0.036% TFA in ACN).
²UPLC-MS: Column C18 Acquity (2.1 mm×50 mm, 1.7 μm, Waters); flux 0.6 mL/min; Gradient 0-100% B in 2 min (A=0.1% formic acid in H₂O and B 0.07% formic acid in ACN).

### Example 12: Ac-D-Asp-D-Ser-D-Glu-D-Asp-D-Asn-D-Ser-D-Asp-D-Glu-D-Glu-D-Asn-NH₂

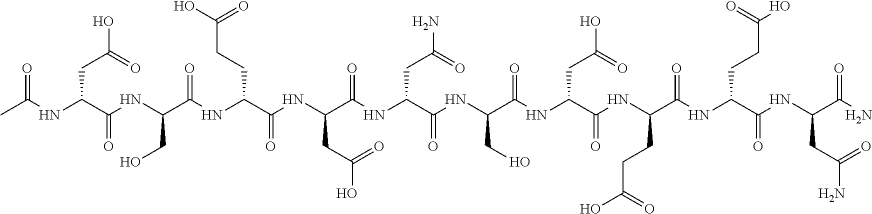 [SMILES: CC(=O)N[C@H](CC(=O)O)C(=O)N[C@H](CO)C(=O)N[C@H](CCC(=O)O)C(=O)N[C@H](CC(=O)O)C(=O)N[C@H](CC(N)=O)C(=O)N[C@H](CO)C(=O)N[C@H](CC(=O)O)C(=O)N[C@H](CCC(=O)O)C(=O)N[C@H](CCC(=O)O)C(=O)N[C@H](CC(N)=O)C(N)=O]

Polymeric support (resin): H-Rink Amide-ChemMatrix®
Used amino acids: Fmoc-D-Asn(Trt)-OH, Fmoc-D-Glu(OtBu)-OH, Fmoc-D-Asp(OtBu)-OH and Fmoc-D-Ser(tBu)-OH.

### General Procedure for Synthesis:

Couplings: the corresponding amino acid (3 eq), Oxyma pure (3 eq) and DIC (3 eq) are dissolved in 2 mL of DMF. Two minutes later, the reaction mixture is added to the resin. For the coupling of the first amino acid to the resin, the reaction is allowed to proceed for 3 h at room temperature with intermittent stirring. For the following amino acids the coupling time is 1 hour. Then, the reaction mixture is filtered off and the resin is washed with DMF (×5) and DCM (×5). Then, a colorimetric test is performed to check the completeness of the reaction. If the reaction is not complete, a recoupling in the same conditions is performed.

Fmoc removal is performed with treatments with a solution of 20% piperidine in DMF (2×5 min, 1×10 min). Washes with DMF (×5) and DCM (×5) are carried out after Fmoc removal. No colorimetric test is performed.

Acetylation: after last amino acid removal, the N-terminal part of the peptide is acetylated with 10 eq of DIEA and 10 eq of acetic anhydride with DCM during 15 minutes.

Cleavage: the resin is washed several times with DCM and dried. The peptide is cleaved from the resin by adding a mixture of 95% TFA, 2.5% TIS and 2.5% water at room temperature for 2 hours under intermittent stirring. The reaction mixture is filtered and rinsed with DCM (×5). All the filtrates are pooled and the solvent is evaporated using a N₂ stream. The crude of synthesis is resuspended in a mixture of water and acetonitrile (1:1), analyzed by HPLC and HPLC-MS. The crude of synthesis is purified by reverse phase chromatography, lyophilized and fully characterized.

Characterization of Example 12 after Purification:

| HPLC retention time | Calculated | | Purity |
|---|---|---|---|
| (min)¹ | MW | Observed MW² | (HPLC, Area/Area) |
| 3.17 | 1194.0 | 1194.2 | >94% |

¹HPLC: Column HPLC Sunfire C18 (4.6 mm×100 mm, 3.5 μm); flux 1 mL/min; Gradient 0-100% B in 8 min (A=0.045% TFA in H₂O and B 0.036% TFA in ACN).
²UPLC-MS: Column C18 Acquity (2.1 mm×50 mm, 1.7 μm, Waters); flux 0.6 mL/min; Gradient 0-100% B in 2 min (A=0.1% formic acid in H₂O and B 0.07% formic acid in ACN).

### Example 13: Ac-D-Asp-D-Asn-D-Asp-D-Ser-D-Glu-D-Asp-D-Asn-D-Ser-D-Asp-D-Glu-D-Glu-D-Ala-NH₂

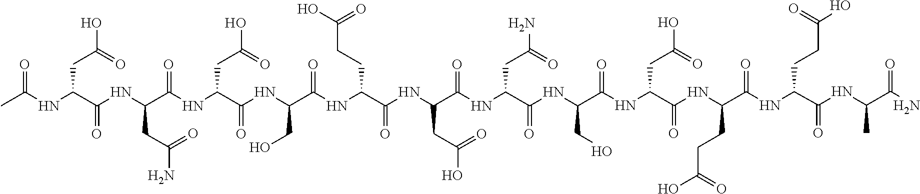

Polymeric support (resin): H-Rink Amide-ChemMatrix®
Used amino acids: Fmoc-D-Ala-OH, Fmoc-D-Asn(Trt)-OH, Fmoc-D-Glu(OtBu)-OH, Fmoc-D-Asp(OtBu)-OH and Fmoc-D-Ser(tBu)-OH.

### General Procedure for Synthesis:

Couplings: the corresponding amino acid (3 eq), Oxyma pure (3 eq) and DIC (3 eq) are dissolved in 2 mL of DMF. Two minutes later, the reaction mixture is added to the resin. For the coupling of the first amino acid to the resin, the reaction is allowed to proceed for 3 h at room temperature with intermittent stirring. For the following amino acids the coupling time is 1 hour. Then, the reaction mixture is filtered off and the resin is washed with DMF (×5) and DCM (×5). Then, a colorimetric test is performed to check the completeness of the reaction. If the reaction is not complete, a recoupling in the same conditions is performed.

Fmoc removal is performed with treatments with a solution of 20% piperidine in DMF (2×5 min, 1×10 min). Washes with DMF (×5) and DCM (×5) are carried out after Fmoc removal. No colorimetric test is performed.

Acetylation: after last amino acid removal, the N-terminal part of the peptide is acetylated with 10 eq of DIEA and 10 eq of acetic anhydride with DCM during 15 minutes.

Cleavage: the resin is washed several times with DCM and dried. The peptide is cleaved from the resin by adding a mixture of 95% TFA, 2.5% TIS and 2.5% water at room temperature for 2 hours under intermittent stirring. The reaction mixture is filtered and rinsed with DCM (×5). All the filtrates are pooled and the solvent is evaporated using a N₂ stream. The crude of synthesis is resuspended in a mixture of water and acetonitrile (1:1), analyzed by HPLC and HPLC-MS. The crude of synthesis is purified by reverse phase chromatography, lyophilized and fully characterized.

Characterization of Example 13 after Purification:

| HPLC retention | | | Purity |
|---|---|---|---|
| time (min)¹ | Calculated MW | Observed MW² | (HPLC, Area/Area) |
| 1.40 | 1380.2 | 1381.1 [M + H]⁺ | >99% |

¹HPLC: Column C18 XSelect CSH (50 mm×4.6 mm, 3.5 μm, 100 Å, Waters); Flow rate 1.6 mL/min; Gradient 0-100% in 3.5 min (A=0.1% TFA in H₂O and B 0.1% TFA in ACN).
²HPLC-MS: Column C18 XSelect CSH (50 mm×4.6 mm, 3.5 μm, 100 Å, Waters); Flow rate 1.6 mL/min T=50° C.; Gradient=5-100% B in 3.5 min (A=0.1% formic acid in H₂O, and B=0.1% formic acid in ACN).

### Example 14: Ac-D-Asn-D-Asp-D-Ser-D-Glu-D-Asp-D-Asn-D-Ser-D-Asp-D-Glu-D-Glu-D-Ala-NH₂

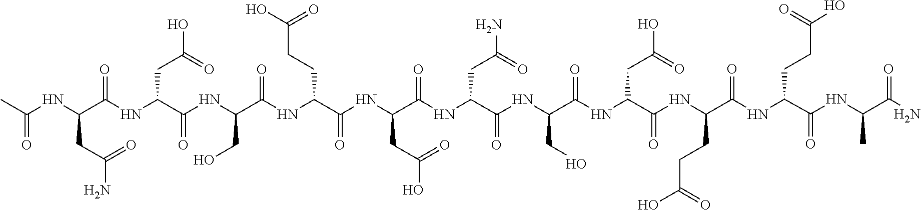

Polymeric support (resin): H-Rink Amide-ChemMatrix®
Used amino acids: Fmoc-D-Ala-OH, Fmoc-D-Asn(Trt)-OH, Fmoc-D-Glu(OtBu)-OH, Fmoc-D-Asp(OtBu)-OH and Fmoc-D-Ser(tBu)-OH.

### General Procedure for Synthesis:

Couplings: the corresponding amino acid (3 eq), Oxyma pure (3 eq) and DIC (3 eq) are dissolved in 2 mL of DMF. Two minutes later, the reaction mixture is added to the resin. For the coupling of the first amino acid to the resin, the reaction is allowed to proceed for 3 h at room temperature with intermittent stirring. For the following amino acids the coupling time is 1 hour. Then, the reaction mixture is filtered off and the resin is washed with DMF (×5) and DCM (×5). Then, a colorimetric test is performed to check the completeness of the reaction. If the reaction is not complete, a recoupling in the same conditions is performed.

Fmoc removal is performed with treatments with a solution of 20% piperidine in DMF (2×5 min, 1×10 min). Washes with DMF (×5) and DCM (×5) are carried out after Fmoc removal. No colorimetric test is performed.

Acetylation: after last amino acid removal, the N-terminal part of the peptide is acetylated with 10 eq of DIEA and 10 eq of acetic anhydride with DCM during 15 minutes.

Cleavage: the resin is washed several times with DCM and dried. The peptide is cleaved from the resin by adding a mixture of 95% TFA, 2.5% TIS and 2.5% water at room temperature for 2 hours under intermittent stirring. The reaction mixture is filtered and rinsed with DCM (×5). All the filtrates are pooled and the solvent is evaporated using a N₂ stream. The crude of synthesis is resuspended in a mixture of water and acetonitrile (1:1), analyzed by HPLC and HPLC-MS. The crude of synthesis is purified by reverse phase chromatography, lyophilized and fully characterized.

Characterization of Example 14 after Purification:

| HPLC retention | | | Purity |
|---|---|---|---|
| time (min)¹ | Calculated MW | Observed MW² | (HPLC, Area/Area) |
| 3.24 | 1265.0 | 1265.3 | >95% |

¹HPLC: Column HPLC Sunfire C18 (4.6 mm×100 mm, 3.5 μm); flux 1 mL/min; Gradient 0-100% B in 8 min (A=0.045% TFA in H₂O and B 0.036% TFA in ACN).
²UPLC-MS: Column C18 Acquity (2.1 mm×50 mm, 1.7 μm, Waters); flux 0.6 mL/min; Gradient 0-100% B in 2 min (A=0.1% formic acid in H₂O and B 0.07% formic acid in ACN).

### Example 15: Ac-D-Asp-D-Ser-D-Glu-D-Asp-D-Asn-D-Ser-D-Asp-D-Glu-D-Glu-D-Ala-NH₂

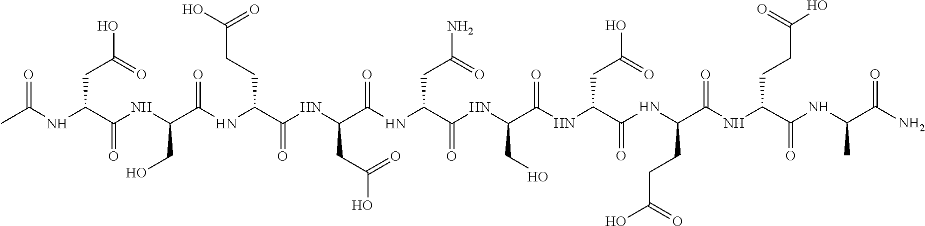 [SMILES: CC(=O)N[C@H](CC(=O)O)C(=O)N[C@H](CO)C(=O)N[C@H](CCC(=O)O)C(=O)N[C@H](CC(=O)O)C(=O)N[C@H](CC(N)=O)C(=O)N[C@H](CO)C(=O)N[C@H](CC(=O)O)C(=O)N[C@H](CCC(=O)O)C(=O)N[C@H](CCC(=O)O)C(=O)N[C@H](C)C(N)=O]

Polymeric support (resin): H-Rink Amide-ChemMatrix®
Used amino acids: Fmoc-D-Ala-OH, Fmoc-D-Asn(Trt)-OH, Fmoc-D-Glu(OtBu)-OH, Fmoc-D-Asp(OtBu)-OH and Fmoc-D-Ser(tBu)-OH.

### General Procedure for Synthesis:

Couplings: the corresponding amino acid (3 eq), Oxyma pure (3 eq) and DIC (3 eq) are dissolved in 2 mL of DMF. Two minutes later, the reaction mixture is added to the resin. For the coupling of the first amino acid to the resin, the reaction is allowed to proceed for 3 h at room temperature with intermittent stirring. For the following amino acids the coupling time is 1 hour. Then, the reaction mixture is filtered off and the resin is washed with DMF (×5) and DCM (×5). Then, a colorimetric test is performed to check the completeness of the reaction. If the reaction is not complete, a recoupling in the same conditions is performed.

Fmoc removal is performed with treatments with a solution of 20% piperidine in DMF (2×5 min, 1×10 min). Washes with DMF (×5) and DCM (×5) are carried out after Fmoc removal. No colorimetric test is performed.

Acetylation: after last amino acid removal, the N-terminal part of the peptide is acetylated with 10 eq of DIEA and 10 eq of acetic anhydride with DCM during 15 minutes.

Cleavage: the resin is washed several times with DCM and dried. The peptide is cleaved from the resin by adding a mixture of 95% TFA, 2.5% TIS and 2.5% water at room temperature for 2 hours under intermittent stirring. The reaction mixture is filtered and rinsed with DCM (×5). All the filtrates are pooled and the solvent is evaporated using a N₂ stream. The crude of synthesis is resuspended in a mixture of water and acetonitrile (1:1), analyzed by HPLC and HPLC-MS. The crude of synthesis is purified by reverse phase chromatography, lyophilized and fully characterized.

Characterization of Example 15 after Purification:

| | | | Purity |
|---|---|---|---|
| HPLC retention | Calculated | | (HPLC, |
| time (min)¹ | MW | Observed MW² | Area/Area) |
| 3.28 | 1151.0 | 1172.9 [M + Na]⁺ | >97% |

¹HPLC: Column HPLC Sunfire C18 (4.6 mm×100 mm, 3.5 μm); flux 1 mL/min; Gradient 0-100% B in 8 min (A=0.045% TFA in H₂O and B 0.036% TFA in ACN).
²UPLC-MS: Column C18 Acquity (2.1 mm×50 mm, 1.7 μm, Waters); flux 0.6 mL/min; Gradient 0-100% B in 2 min (A=0.1% formic acid in H₂O and B 0.07% formic acid in ACN).

### Example 16: Ac-D-Asp-D-Ser-D-Glu-D-Asp-D-Asn-D-Ala-D-Asp-D-Glu-D-Glu-D-Ala-NH₂

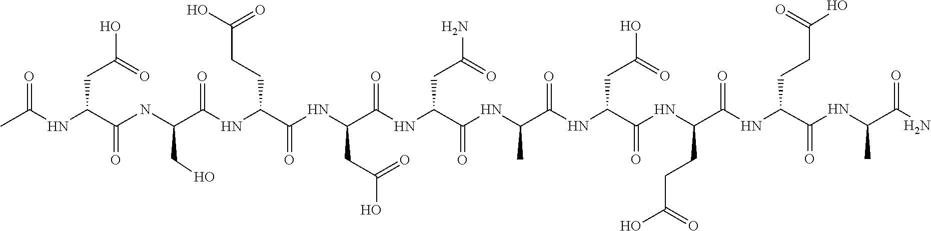 [SMILES: CC(=O)N[C@H](CC(=O)O)C(=O)N[C@H](CO)C(=O)N[C@H](CCC(=O)O)C(=O)N[C@H](CC(=O)O)C(=O)N[C@H](CC(N)=O)C(=O)N[C@H](C)C(=O)N[C@H](CC(=O)O)C(=O)N[C@H](CCC(=O)O)C(=O)N[C@H](CCC(=O)O)C(=O)N[C@H](C)C(N)=O]

Polymeric support (resin): H-Rink Amide-ChemMatrix®
Used amino acids: Fmoc-D-Ala-OH, Fmoc-D-Asn(Trt)-OH, Fmoc-D-Glu(OtBu)-OH, Fmoc-D-Asp(OtBu)-OH and Fmoc-D-Ser(tBu)-OH.

### General Procedure for Synthesis:

Couplings: the corresponding amino acid (3 eq), Oxyma pure (3 eq) and DIC (3 eq) are dissolved in 2 mL of DMF. Two minutes later, the reaction mixture is added to the resin. For the coupling of the first amino acid to the resin, the reaction is allowed to proceed for 3 h at room temperature with intermittent stirring. For the following amino acids the coupling time is 1 hour. Then, the reaction mixture is filtered off and the resin is washed with DMF (×5) and DCM (×5). Then, a colorimetric test is performed to check the completeness of the reaction. If the reaction is not complete, a recoupling in the same conditions is performed.

Fmoc removal is performed with treatments with a solution of 20% piperidine in DMF (2×5 min, 1×10 min). Washes with DMF (×5) and DCM (×5) are carried out after Fmoc removal. No colorimetric test is performed.

Acetylation: after last amino acid removal, the N-terminal part of the peptide is acetylated with 10 eq of DIEA and 10 eq of acetic anhydride with DCM during 15 minutes.

Cleavage: the resin is washed several times with DCM and dried. The peptide is cleaved from the resin by adding a mixture of 95% TFA, 2.5% TIS and 2.5% water at room temperature for 2 hours under intermittent stirring. The reaction mixture is filtered and rinsed with DCM (×5). All the filtrates are pooled and the solvent is evaporated using a N₂ stream. The crude of synthesis is resuspended in a mixture of water and acetonitrile (1:1), analyzed by HPLC and HPLC-MS. The crude of synthesis is purified by reverse phase chromatography, lyophilized and fully characterized.

Characterization of Example 16 after Purification:

| | | | Purity |
|---|---|---|---|
| HPLC retention | Calculated | | (HPLC, |
| time (min)¹ | MW | Observed MW² | Area/Area) |
| 1.34 | 1135.0 | 1135.6 [M + H]⁺ | >90% |
| | | 568.6 [M + H]²⁺ | |

¹HPLC: Column C18 XSelect CSH (50 mm×4.6 mm, 3.5 μm, 100 Å, Waters); Flow rate 1.6 mL/min; Gradient 0-100% in 3.5 min (A=0.1% TFA in H₂O and B 0.1% TFA in ACN).
²HPLC-MS: Column C18 XSelect CSH (50 mm×4.6 mm, 3.5 μm, 100 Å, Waters); Flow rate 1.6 mL/min T=50° C.; Gradient=5-100% B in 3.5 min (A=0.1% formic acid in H₂O, and B=0.1% formic acid in ACN).

### Example 17: Ac-D-Asp-D-Ala-D-Glu-D-Asp-D-Asn-D-Ala-D-Asp-D-Glu-D-Glu-D-Ala-NH₂

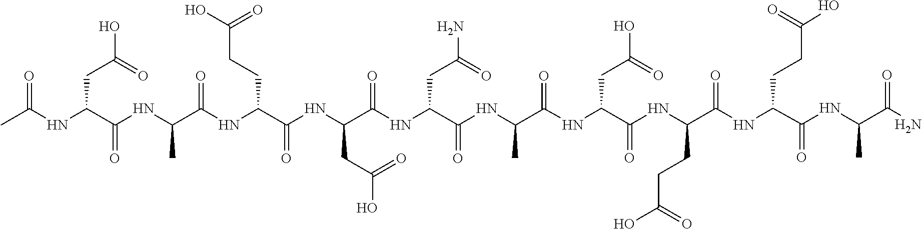 [SMILES: CC(=O)N[C@H](CC(=O)O)C(=O)N[C@H](C)C(=O)N[C@H](CCC(=O)O)C(=O)N[C@H](CC(=O)O)C(=O)N[C@H](CC(N)=O)C(=O)N[C@H](C)C(=O)N[C@H](CC(=O)O)C(=O)N[C@H](CCC(=O)O)C(=O)N[C@H](CCC(=O)O)C(=O)N[C@H](C)C(N)=O]

Polymeric support (resin): H-Rink Amide-ChemMatrix®
Used amino acids: Fmoc-D-Ala-OH, Fmoc-D-Asn(Trt)-OH, Fmoc-D-Glu(OtBu)-OH and Fmoc-D-Asp(OtBu)-OH.

### General Procedure for Synthesis:

Couplings: the corresponding amino acid (3 eq), Oxyma pure (3 eq) and DIC (3 eq) are dissolved in 2 mL of DMF. Two minutes later, the reaction mixture is added to the resin. For the coupling of the first amino acid to the resin, the reaction is allowed to proceed for 3 h at room temperature with intermittent stirring. For the following amino acids the coupling time is 1 hour. Then, the reaction mixture is filtered off and the resin is washed with DMF (×5) and DCM (×5). Then, a colorimetric test is performed to check the completeness of the reaction. If the reaction is not complete, a recoupling in the same conditions is performed.

Fmoc removal is performed with treatments with a solution of 20% piperidine in DMF (2×5 min, 1×10 min). Washes with DMF (×5) and DCM (×5) are carried out after Fmoc removal. No colorimetric test is performed.

Acetylation: after last amino acid removal, the N-terminal part of the peptide is acetylated with 10 eq of DIEA and 10 eq of acetic anhydride with DCM during 15 minutes.

Cleavage: the resin is washed several times with DCM and dried. The peptide is cleaved from the resin by adding a mixture of 95% TFA, 2.5% TIS and 2.5% water at room temperature for 2 hours under intermittent stirring. The reaction mixture is filtered and rinsed with DCM (×5). All the filtrates are pooled and the solvent is evaporated using a N₂ stream. The crude of synthesis is resuspended in a mixture of water and acetonitrile (1:1), analyzed by HPLC and HPLC-MS. The crude of synthesis is purified by reverse phase chromatography, lyophilized and fully characterized.

Characterization of Example 17 after Purification:

| HPLC retention | | | Purity |
|---|---|---|---|
| time (min)¹ | Calculated MW | Observed MW² | (HPLC, Area/Area) |
| 1.45 | 1119.0 | 1119.8 [M + H]⁺ | >94% |

¹HPLC: Column C18 XSelect CSH (50 mm×4.6 mm, 3.5 μm, 100 Å, Waters); Flow rate 1.6 mL/min; Gradient 0-100% in 3.5 min (A=0.1% TFA in H₂O and B 0.1% TFA in ACN).
²HPLC-MS: Column C18 XSelect CSH (50 mm×4.6 mm, 3.5 μm, 100 Å, Waters); Flow rate 1.6 mL/min T=50° C.; Gradient=5-100% B in 3.5 min (A=0.1% formic acid in H₂O, and B=0.1% formic acid in ACN).

### Example 18: Ac-D-Asp-D-Ser-D-Glu-D-Asp-D-Asn-D-Lys-D-Asp-D-Glu-D-Glu-D-Asn-NH₂

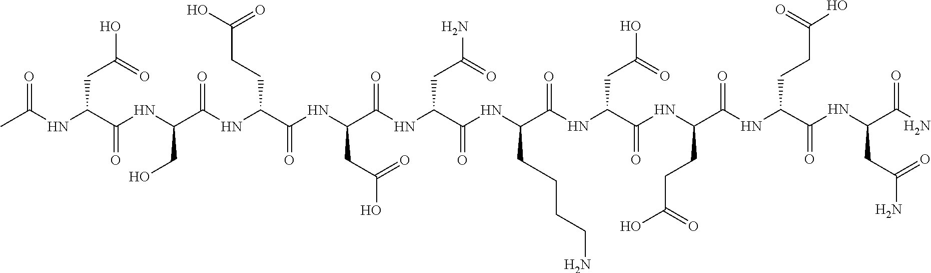 [SMILES: CC(=O)N[C@H](CC(=O)O)C(=O)N[C@H](CO)C(=O)N[C@H](CCC(=O)O)C(=O)N[C@H](CC(=O)O)C(=O)N[C@H](CC(N)=O)C(=O)N[C@H](CCCCN)C(=O)N[C@H](CC(=O)O)C(=O)N[C@H](CCC(=O)O)C(=O)N[C@H](CCC(=O)O)C(=O)N[C@H](CC(N)=O)C(N)=O]

Polymeric support (resin): H-Rink Amide-ChemMatrix®
Used amino acids: Fmoc-D-Asn(Trt)-OH, Fmoc-D-Glu(OtBu)-OH, Fmoc-D-Asp(OtBu)-OH, Fmoc-D-Lys(Boc)-OH and Fmoc-D-Ser(tBu)-OH.

### General Procedure for Synthesis:

Couplings: the corresponding amino acid (3 eq), Oxyma pure (3 eq) and DIC (3 eq) are dissolved in 2 mL of DMF. Two minutes later, the reaction mixture is added to the resin. For the coupling of the first amino acid to the resin, the reaction is allowed to proceed for 3 h at room temperature with intermittent stirring. For the following amino acids the coupling time is 1 hour. Then, the reaction mixture is filtered off and the resin is washed with DMF (×5) and DCM (×5). Then, a colorimetric test is performed to check the completeness of the reaction. If the reaction is not complete, a recoupling in the same conditions is performed.

Fmoc removal is performed with treatments with a solution of 20% piperidine in DMF (2×5 min, 1×10 min). Washes with DMF (×5) and DCM (×5) are carried out after Fmoc removal. No colorimetric test is performed.

Acetylation: after last amino acid removal, the N-terminal part of the peptide is acetylated with 10 eq of DIEA and 10 eq of acetic anhydride with DCM during 15 minutes.

Cleavage: the resin is washed several times with DCM and dried. The peptide is cleaved from the resin by adding a mixture of 95% TFA, 2.5% TIS and 2.5% water at room temperature for 2 hours under intermittent stirring. The reaction mixture is filtered and rinsed with DCM (×5). All the filtrates are pooled and the solvent is evaporated using a N₂ stream. The crude of synthesis is resuspended in a mixture of water and acetonitrile (1:1), analyzed by HPLC and HPLC-MS. The crude of synthesis is purified by reverse phase chromatography, lyophilized and fully characterized.

Characterization of Example 18 after Purification:

| | | | Purity |
|---|---|---|---|
| HPLC retention | Calculated | | (HPLC, |
| time (min)¹ | MW | Observed MW² | Area/Area) |
| 1.32 | 1235.1 | 1236.0 [M + H]⁺ | >95% |

¹HPLC: Column C18 XSelect CSH (50 mm×4.6 mm, 3.5 μm, 100 Å, Waters); Flow rate 1.6 mL/min; Gradient 0-100% in 3.5 min (A=0.1% TFA in H₂O and B 0.1% TFA in ACN).
²HPLC-MS: Column C18 XSelect CSH (50 mm×4.6 mm, 3.5 μm, 100 Å, Waters); Flow rate 1.6 mL/min T=50° C.; Gradient=5-100% B in 3.5 min (A=0.1% formic acid in H₂O, and B=0.1% formic acid in ACN).

### Example 19: Ac-D-Asp-D-Ser-D-Glu-D-Asp-D-Asn-D-Lys-D-Asp-D-Glu-D-Glu-D-Ala-NH₂

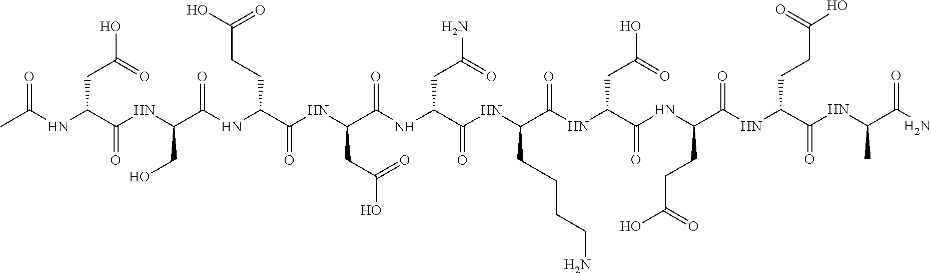 [SMILES: CC(=O)N[C@H](CC(=O)O)C(=O)N[C@H](CO)C(=O)N[C@H](CCC(=O)O)C(=O)N[C@H](CC(=O)O)C(=O)N[C@H](CC(N)=O)C(=O)N[C@H](CCCCN)C(=O)N[C@H](CC(=O)O)C(=O)N[C@H](CCC(=O)O)C(=O)N[C@H](CCC(=O)O)C(=O)N[C@H](C)C(N)=O]

Polymeric support (resin): H-Rink Amide-ChemMatrix®
Used amino acids: Fmoc-D-Ala-OH, Fmoc-D-Asn(Trt)-OH, Fmoc-D-Glu(OtBu)-OH, Fmoc-D-Asp(OtBu)-OH, Fmoc-D-Lys(Boc)-OH and Fmoc-D-Ser(tBu)-OH.

### General Procedure for Synthesis:

Couplings: the corresponding amino acid (3 eq), Oxyma pure (3 eq) and DIC (3 eq) are dissolved in 2 mL of DMF. Two minutes later, the reaction mixture is added to the resin. For the coupling of the first amino acid to the resin, the reaction is allowed to proceed for 3 h at room temperature with intermittent stirring. For the following amino acids the coupling time is 1 hour. Then, the reaction mixture is filtered off and the resin is washed with DMF (×5) and DCM (×5). Then, a colorimetric test is performed to check the completeness of the reaction. If the reaction is not complete, a recoupling in the same conditions is performed.

Fmoc removal is performed with treatments with a solution of 20% piperidine in DMF (2×5 min, 1×10 min). Washes with DMF (×5) and DCM (×5) are carried out after Fmoc removal. No colorimetric test is performed.

Acetylation: after last amino acid removal, the N-terminal part of the peptide is acetylated with 10 eq of DIEA and 10 eq of acetic anhydride with DCM during 15 minutes.

Cleavage: the resin is washed several times with DCM and dried. The peptide is cleaved from the resin by adding a mixture of 95% TFA, 2.5% TIS and 2.5% water at room temperature for 2 hours under intermittent stirring. The reaction mixture is filtered and rinsed with DCM (×5). All the filtrates are pooled and the solvent is evaporated using a N₂ stream. The crude of synthesis is resuspended in a mixture of water and acetonitrile (1:1), analyzed by HPLC and HPLC-MS. The crude of synthesis is purified by reverse phase chromatography, lyophilized and fully characterized.

Characterization of Example 19 after Purification:

| | | | Purity |
|---|---|---|---|
| HPLC retention | Calculated | | (HPLC, |
| time (min)¹ | MW | Observed MW² | Area/Area) |
| 1.42 | 1192.1 | 1193.0 [M + H]⁺ | >92% |

¹HPLC: Column C18 XSelect CSH (50 mm×4.6 mm, 3.5 μm, 100 Å, Waters); Flow rate 1.6 mL/min; Gradient 0-100% in 3.5 min (A=0.1% TFA in H₂O and B 0.1% TFA in ACN).
²HPLC-MS: Column C18 XSelect CSH (50 mm×4.6 mm, 3.5 μm, 100 Å, Waters); Flow rate 1.6 mL/min T=50° C.; Gradient=5-100% B in 3.5 min (A=0.1% formic acid in H₂O, and B=0.1% formic acid in ACN).

### Experiments:

Determination of Inhibitory Effect of Novel Compounds on Human LDL Aggregation

The efficacy of the novel compounds to inhibit the aggregation of LDL has been tested in two parallel turbidimetric assays differing from each other only in the enzyme used to induce LDL aggregation, namely sphingomyelinase (SMase) and phospholipase A2 (PLA2), respectively.

LDL Isolation and Purification

Human LDL (d_{1.019}-d_{1.063} g/mL) were obtained from pooled normolipemic human plasma by sequential ultracentrifugation in a KBr density gradient. Briefly, VLDL were first discarded after spinning plasma at 36.000 rpm for 18 h at 4° C., VLDL-free plasma was layered with 1.063 g/mL KBr solution and centrifuged at 36,000 rpm for 18 h at 4° C. LDL were dialyzed against 0.02 M Tris, 0.15 M NaCl, 1 mM EDTA, pH 7.5 for 18 hours, and then against normal saline for 2 hours. Finally, isolated LDL were filter-sterilized. Protein concentration was determined using a colorimetric assay.

Anti-LDL Aggregation Assay

Human LDL particles (1.44 mg/mL) were incubated with 40 U/L of Bacillus cereus SMase or with 50 μg/L of type II secretory PLA2 from honey bee venom in 20 mM Tris buffer (pH 7.0) containing 150 mM NaCl, 2 mM CaCl₂), and MgCl₂ at 37° C. LDL incubation with SMase and PLA2 was performed at peptide concentration of 10 μM (ratio compound/ApoB-100: 4.7/1) for 18 hours. LDL lipolysis was stopped by addition of EDTA (final concentration 10 mM).

The efficiency of each novel compound to inhibit LDL aggregation induced by SMase and PLA2 was estimated by turbidimetry measuring the absorbance at a wavelength of 405 nm. Thus, the inhibitory activity was calculated according to Equation I.

$$Inhibition activity = [ 1 - ( a - b / c - b ) ] * 100 \qquad ( Equation I )$$

wherein:

a corresponds to the absorbance value in the presence of nLDL particles, LDL aggregating enzyme (i.e., SMase or PLA2) and test compound;

b corresponds to the absorbance value in the presence of only nLDL particles;

c corresponds to the absorbance value in the presence of nLDL particles and LDL aggregating enzyme (i.e., SMase or PLA2);

The inhibitory activity of each novel compound on LDL aggregation induced by either SMase and PLA2 is detailed in Table 1.

TABLE 1
Inhibition of LDL aggregation

| Example | SMase-treated LDL | PLA2-treated LDL |
|---|---|---|
| 1 | 92.5 | 77.5 |
| 2 | 80.7 | 87.7 |
| 3 | 90.0 | 37.2 |
| 4 | 91.5 | 32.6 |
| 5 | 90.1 | 20.7 |
| 6 | 85.0 | 15.3 |
| 7 | 44.7 | −1.9 |
| 8 | 64.2 | 3.1 |
| 9 | 91.0 | 36.4 |
| 10 | 96.0 | 94.0 |
| 11 | 94.8 | 95.2 |
| 12 | 93.3 | 94.3 |
| 13 | 95.5 | 95.9 |
| 14 | 68.3 | 74.0 |
| 15 | 96.0 | 95.9 |
| 16 | 94.0 | 61.9 |
| 17 | 95.2 | 66.1 |
| 18 | 85.1 | 55.1 |
| 19 | 87.5 | 46.2 |

### Determination of Inhibitory Effect of Novel Compounds on the Intracellular Cholesterol Accumulation Induced by SMase

Human coronary vascular smooth muscle cells (hVSMC) exposed to LDL (nLDL or SMase-LDL) showed similar free cholesterol (FC) levels than hVSMC unexposed to LDL, indicating that LDL did not alter FC content in these cells. Conversely, intracellular cholesteryl esters (CE) detected in these cells upon exposure to LDL derives exclusively from CE supplied by LDL as hcVSMC unexposed to LDL did not have intracellular cholesteryl esters (CE).

Thus, the inhibitory effect of the novel compounds on the intracellular cholesterol accumulation has been analysed in terms of decrease in the ratio of intracellular CE and free cholesterol FC content of hcVSMC exposed to LDL and SMase-LDL.

Culture of Human Coronary Vascular Smooth Cells (hVSMC)

All cells used were from a unique batch in order to prevent variability from cell origin. Cell quiescence was induced by maintaining the cell culture for 24 hours in a medium. Serum-deprived cells between passages 4 and 8 were used for experiments. VSMCs at these passages appeared as a relatively homogeneous population, showing a hill-and-valley confluence pattern. Cell monolayers were grown in vascular cell basal medium supplemented with vascular smooth muscle growth kit components. Quiescent cells were 2-hours exposed to LDL treated with SMase (in absence or presence of peptides) overnight at a concentration of 10 μM previously checked its aggregation by a turbidimetry test. Cells were then either collected for lipid extraction or processed for confocal microscopy analysis.

### Lipid Extraction and Determination of Free and Cholesteryl Ester Content

Following the lipoprotein incubation period, hcVSMC were exhaustively washed (twice with PBS, twice with PBS supplemented with 1% BSA, and once with PBS supplemented with both 1% BSA and 100 U/mL heparin), before they were harvested into 1 mL of 0.15 M NaOH. Lipids were extracted using the Bligh and Dyer method. The lipid extract was redissolved in dichloromethane, applied to silica gel plates, and separated by thin layer chromatography. A mixture of cholesterol and cholesterol palmitate, were also run as standards. Heptane or a solvent combination of heptane/diethylether/acetic acid (74:21:4, v/v/v) were used as chromatographic mobile phase. After lipid separation, the plates were dried and stained as previously reported. Finally, the spots corresponding to cholesteryl ester (CE) and free cholesterol (FC) were quantitated by densitometry.

The efficacy of each novel compound to decrease the ratio of intracellular cholesteryl esters (CE) and free cholesterol (FC) content of hcVSMC exposed to LDL and SMase-LDL was calculated according to Equation II.

$$Efficacy = [ 1 - ( a - b / c - b ) ] * 100 \qquad ( Equation II )$$

wherein:

a corresponds to the CE/FC ratio in hcVSMC exposed to SMase-LDL and in the presence of the test compound.

b corresponds to the CE/FC ratio in hcVSMC exposed to nLDL in the absence of the test compound.

c corresponds to the CE/FC ratio in hcVSMC exposed to SMase-LDL in the absence of the test compound.

The inhibitory effect of each novel compound on the intracellular cholesterol accumulation induced by SMase is detailed in Table 2.

TABLE 2
inhibitory effect of each novel compound on the intracellular cholesterol
accumulation induced by SMase. Efficacy is measured as the decrease
in the ratio between intracellular cholesteryl esters (CE) and
free cholesterol (FC) content of human coronary vascular smooth
muscle cells (hcVSMC) exposed to LDL and SMase-LDL

| | CE/FC ratio |
|---|---|
| Example | decrease (%) |
| 1 | 76.5 |
| 2 | 58.8 |
| 3 | N/A |
| 4 | N/A |
| 5 | N/A |
| 6 | 58.8 |
| 7 | N/A |
| 8 | N/A |
| 9 | 58.8 |
| 10 | 78.4 |
| 11 | 76.5 |
| 12 | 76.5 |
| 13 | 80.4 |
| 14 | 62.7 |
| 15 | 90.2 |
| 16 | 69.5 |
| 17 | 67.4 |
| 18 | 47.6 |
| 19 | 17.7 |

The following are particular embodiments of the present invention:

Embodiment 1: A compound of formula (I):

R¹-AA¹-AA²-AA³-AA⁴-AA⁵-D-Glu-D-Asp-AA⁶-AA⁷-AA⁸-D-Glu-D-Glu-AA⁹-NH₂ (I)

wherein
R¹ is a C₂₋₄ acyl group, preferably acetyl,
AA¹ is either absent or is Gly
AA² is either absent or is D-Asp
AA³ is either absent or is D-Asn
AA⁴ is either absent or is D-Asp
AA⁵ is selected from the group consisting of D-Ser and D-Ala
AA⁶ is selected from the group consisting of D-Asn and D-Gln
AA⁷ is selected from the group consisting of D-Ala, D-Arg, D-Lys and D-Ser
AA⁸ is selected from the group consisting of D-Asp and D-Glu
AA⁹ is selected from the group consisting of D-Ala, D-Arg, D-Asn, D-Gln, D-Leu and D-Trp
   or a salt or solvate thereof.

Embodiment 2: A compound according to Embodiment 1 wherein R¹ is acetyl.

Embodiment 3: A compound according to anyone of Embodiments 1 to 2 wherein AA¹ is absent.

Embodiment 4: A compound according to anyone of Embodiments 1 to 3 wherein AA² is absent.

Embodiment 5: A compound according to anyone of Embodiments 1 to 4 wherein AA³ is absent.

Embodiment 6: A compound according to Embodiment 1 wherein -AA¹-AA²-AA³-AA⁴-AA⁵- is either absent or is selected from the group consisting of -D-Gly-D-Asp-D-Asn-D-Asp-D-Ser, -D-Asp-D-Asn-D-Asp-D-Ser- and -D-Asn-D-Asp-D-Ser-, -D-Asp-D-Ser and -D-Asp-D-Ala-.

Embodiment 7: A compound according to anyone of Embodiments 1 to 6 wherein AA⁵ is D-Ser.

Embodiment 8: A compound according to anyone of Embodiments 1 to 7 wherein -AA⁶-AA⁷-AA⁸- is selected from the group consisting of -D-Asn-D-Ser-D-Asp-, -D-Asn-D-Ala-D-Asp-, -D-Gln-D-Ser-D-Glu-, -D-Gln-D-Ala-D-Glu-, -D-Asn-D-Arg-D-Asp- and -D-Asn-D-Lys-D-Asp-.

Embodiment 9: A compound according to Embodiment 8 wherein -AA⁶-AA⁷-AA⁸- is -D-Asn-D-Ser-D-Asp-.

Embodiment 10: A compound according to anyone of Embodiments 1 to 9 wherein -AA⁹- is selected from the group consisting of D-Ala and D-Asn.

Embodiment 11: A compound according to Embodiment 1 selected from the group consisting of:
Ac-Gly-D-Asp-D-Asn-D-Asp-D-Ser-D-Glu-D-Asp-D-Asn-D-Ser-D-Asp-D-Glu-D-Glu-D-Asn-NH₂
Ac-D-Ser-D-Glu-D-Asp-D-Asn-D-Ser-D-Asp-D-Glu-D-Glu-D-Asn-NH₂
Ac-D-Ser-D-Glu-D-Asp-D-Asn-D-Ala-D-Asp-D-Glu-D-Glu-D-Ala-NH₂
Ac-D-Ser-D-Glu-D-Asp-D-Gln-D-Ser-D-Glu-D-Glu-D-Glu-D-Gln-NH₂
Ac-D-Ser-D-Glu-D-Asp-D-Gln-D-Ala-D-Glu-D-Glu-D-Glu-D-Ala-NH₂
Ac-D-Ser-D-Glu-D-Asp-D-Asn-D-Arg-D-Asp-D-Glu-D-Glu-D-Arg-NH₂
Ac-D-Ser-D-Glu-D-Asp-D-Asn-D-Arg-D-Asp-D-Glu-D-Glu-D-Leu-NH₂
Ac-D-Ser-D-Glu-D-Asp-D-Asn-D-Arg-D-Asp-D-Glu-D-Glu-D-Trp-NH₂
Ac-D-Ser-D-Glu-D-Asp-D-Asn-D-Lys-D-Asp-D-Glu-D-Glu-D-Asn-NH₂
Ac-D-Asp-D-Asn-D-Asp-D-Ser-D-Glu-D-Asp-D-Asn-D-Ser-D-Asp-D-Glu-D-Glu-D-Asn-NH₂
Ac-D-Asn-D-Asp-D-Ser-D-Glu-D-Asp-D-Asn-D-Ser-D-Asp-D-Glu-D-Glu-D-Asn-NH₂
Ac-D-Asp-D-Ser-D-Glu-D-Asp-D-Asn-D-Ser-D-Asp-D-Glu-D-Glu-D-Asn-NH₂
Ac-D-Asp-D-Asn-D-Asp-D-Ser-D-Glu-D-Asp-D-Asn-D-Ser-D-Asp-D-Glu-D-Glu-D-Ala-NH₂
Ac-D-Asn-D-Asp-D-Ser-D-Glu-D-Asp-D-Asn-D-Ser-D-Asp-D-Glu-D-Glu-D-Ala-NH₂
Ac-D-Asp-D-Ser-D-Glu-D-Asp-D-Asn-D-Ser-D-Asp-D-Glu-D-Glu-D-Ala-NH₂
Ac-D-Asp-D-Ser-D-Glu-D-Asp-D-Asn-D-Ala-D-Asp-D-Glu-D-Glu-D-Ala-NH₂
Ac-D-Asp-D-Ala-D-Glu-D-Asp-D-Asn-D-Ala-D-Asp-D-Glu-D-Glu-D-Ala-NH₂
Ac-D-Asp-D-Ser-D-Glu-D-Asp-D-Asn-D-Lys-D-Asp-D-Glu-D-Glu-D-Asn-NH₂
Ac-D-Asp-D-Ser-D-Glu-D-Asp-D-Asn-D-Lys-D-Asp-D-Glu-D-Glu-D-Ala-NH₂
   or a pharmaceutically acceptable salt or solvate thereof.

Embodiment 12: Pharmaceutical compositions comprising a compound according to anyone of Embodiments 1 to 11 and a pharmaceutically acceptable carrier, adjuvant or vehicle.

Embodiment 13: A compound according to anyone of Embodiments 1 to 11 for use as a medicament.

Embodiment 14: A compound according to anyone of Embodiments 1 to 11 for use in the prevention and/or treatment of conditions wherein decrease of vascular cholesterol accumulation, inhibition of LDL aggregation and/or prevention of aggregated LDL (agLDL) internalization is useful.

Embodiment 15: A compound for use according to Embodiment 14 wherein the condition is selected from the group consisting of atherosclerosis, coronary artery disease, stroke, peripheral artery disease, angina pectoris, thrombosis, hyperlipidemia, hyperlipoproteinemia type II, familial hypercholesterolemia, familial combined hyperlipidemia, type II diabetes, hypothyroidism, Cushing's syndrome and obesity.

1. A compound of formula (I):
R¹-AA¹-AA²-AA³-AA⁴-AA⁵-D-Glu-D-Asp-AA⁶-AA⁷-AA⁸-D-Glu-D-Glu-AA⁹-NH₂ (I)
wherein
R¹ is a C24 acyl group,
AA¹ is either absent or is Gly
AA² is either absent or is D-Asp
AA³ is either absent or is D-Asn
AA⁴ is either absent or is D-Asp
AA⁵ is selected from the group consisting of D-Ser and D-Ala
AA⁶ is selected from the group consisting of D-Asn and D-Gln
AA⁷ is selected from the group consisting of D-Ala, D-Arg, D-Lys and D-Ser
AA⁸ is selected from the group consisting of D-Asp and D-Glu
AA⁹ is selected from the group consisting of D-Ala, D-Arg, D-Asn, D-Gln, D-Leu and D-Trp or a salt or solvate thereof.

2. The compound according to claim 1 wherein R¹ is acetyl.

3. The compound according to claim 1 wherein AA¹ is absent.

4. The compound according to claim 1 wherein AA² is absent.

5. The compound according to claim 1 wherein AA³ is absent.

6. The compound according to claim 1 wherein -AA¹-AA²-AA³-AA⁴-AA⁵- is either absent or is selected from the group consisting of -Gly-D-Asp-D-Asn-D-Asp-D-Ser, -D-Asp-D-Asn-D-Asp-D-Ser- and -D-Asn-D-Asp-D-Ser-, -D-Asp-D-Ser and -D-Asp-D-Ala-.

7. The compound according to claim 1 wherein AA⁵ is D-Ser.

8. The compound according to claim 1 wherein -AA⁶-AA⁷-AA⁸- is selected from the group consisting of -D-Asn-D-Ser-D-Asp-, -D-Asn-D-Ala-D-Asp-, -D-Gln-D-Ser-D-Glu-, -D-Gln-D-Ala-D-Glu-, -D-Asn-D-Arg-D-Asp- and -D-Asn-D-Lys-D-Asp-.

9. The compound according to claim 8 wherein -AA⁶-AA⁷-AA⁸- is -D-Asn-D-Ser-D-Asp-.

10. The compound according to claim 1 wherein -AA⁹- is selected from the group consisting of D-Ala and D-Asn.

11. The compound according to claim 1 selected from the group consisting of:
Ac-Gly-D-Asp-D-Asn-D-Asp-D-Ser-D-Glu-D-Asp-D-Asn-D-Ser-D-Asp-D-Glu-D-Glu-D-Asn-NH₂
Ac-D-Ser-D-Glu-D-Asp-D-Asn-D-Ser-D-Asp-D-Glu-D-Glu-D-Asn-NH₂
Ac-D-Ser-D-Glu-D-Asp-D-Asn-D-Ala-D-Asp-D-Glu-D-Glu-D-Ala-NH₂
Ac-D-Ser-D-Glu-D-Asp-D-Gln-D-Ser-D-Glu-D-Glu-D-Glu-D-Gln-NH₂
Ac-D-Ser-D-Glu-D-Asp-D-Gln-D-Ala-D-Glu-D-Glu-D-Glu-D-Ala-NH₂
Ac-D-Ser-D-Glu-D-Asp-D-Asn-D-Arg-D-Asp-D-Glu-D-Glu-D-Arg-NH₂
Ac-D-Ser-D-Glu-D-Asp-D-Asn-D-Arg-D-Asp-D-Glu-D-Glu-D-Leu-NH₂
Ac-D-Ser-D-Glu-D-Asp-D-Asn-D-Arg-D-Asp-D-Glu-D-Glu-D-Trp-NH₂
Ac-D-Ser-D-Glu-D-Asp-D-Asn-D-Lys-D-Asp-D-Glu-D-Glu-D-Asn-NH₂
Ac-D-Asp-D-Asn-D-Asp-D-Ser-D-Glu-D-Asp-D-Asn-D-Ser-D-Asp-D-Glu-D-Glu-D-Asn-NH₂
Ac-D-Asn-D-Asp-D-Ser-D-Glu-D-Asp-D-Asn-D-Ser-D-Asp-D-Glu-D-Glu-D-Asn-NH₂
Ac-D-Asp-D-Ser-D-Glu-D-Asp-D-Asn-D-Ser-D-Asp-D-Glu-D-Glu-D-Asn-NH₂
Ac-D-Asp-D-Asn-D-Asp-D-Ser-D-Glu-D-Asp-D-Asn-D-Ser-D-Asp-D-Glu-D-Glu-D-Ala-NH₂
Ac-D-Asn-D-Asp-D-Ser-D-Glu-D-Asp-D-Asn-D-Ser-D-Asp-D-Glu-D-Glu-D-Ala-NH₂
Ac-D-Asp-D-Ser-D-Glu-D-Asp-D-Asn-D-Ser-D-Asp-D-Glu-D-Glu-D-Ala-NH₂
Ac-D-Asp-D-Ser-D-Glu-D-Asp-D-Asn-D-Ala-D-Asp-D-Glu-D-Glu-D-Ala-NH₂
Ac-D-Asp-D-Ala-D-Glu-D-Asp-D-Asn-D-Ala-D-Asp-D-Glu-D-Glu-D-Ala-NH₂
Ac-D-Asp-D-Ser-D-Glu-D-Asp-D-Asn-D-Lys-D-Asp-D-Glu-D-Glu-D-Asn-NH₂
Ac-D-Asp-D-Ser-D-Glu-D-Asp-D-Asn-D-Lys-D-Asp-D-Glu-D-Glu-D-Ala-NH₂
and a pharmaceutically acceptable salt or solvate thereof.

12. A pharmaceutical composition comprising a compound according to claim 1 and a pharmaceutically acceptable carrier, adjuvant or vehicle.

13.-17. (canceled)

18. A method for the prevention and/or treatment of conditions wherein decrease of vascular cholesterol accumulation, inhibition of LDL aggregation; prevention of aggregated LDL (agLDL) internalization and/or inhibition or reduction of the formation of VSMC foam cells is useful in a subject in need thereof comprising the administration of a compound according to claim 1.

19. A method according to claim 18 wherein the condition is selected from the group consisting of atherosclerosis, coronary artery disease, stroke, peripheral artery disease, angina pectoris, thrombosis, hyperlipidemia, hyperlipoproteinemia type II, familial hypercholesterolemia, familial combined hyperlipidemia, type II diabetes, hypothyroidism, Cushing's syndrome and obesity.
